(19) United States
(12) Patent Application Publication
(10) Pub. No.: US 2026/0085036 A1
(43) Pub. Date: Mar. 26, 2026

(54) NOVEL NOOTROPIC PRODRUGS OF HENETHYLAMINE

(71) Applicant: MiHKAL GmbH, Stansstad (CH)

(72) Inventor: Matthias GRILL, Allschwil (CH)

(73) Assignee: MiHKAL GmbH, Stansstad (CH)

(21) Appl. No.: 19/110,250

(22) PCT Filed: Sep. 12, 2023
(86) PCT No.: PCT/EP2023/075049

§ 371 Date: Mar. 10, 2025

(30) Foreign Application Priority Data
Sep. 12, 2022 (EP) 22195234.4
Nov. 4, 2022 (EP) 22205644.2

(51) Int. Cl. C07C 271/12; A61K 31/15; A61K 31/277; A61K 31/325; A61K 31/39; A61K 31/405; C07C 251/24; C07C 255/43; C07C 271/16; C07D 327/04; C07D 411/12

(52) U.S. Cl. CPC C07C 271/12; A61K 31/15; A61K 31/277; A61K 31/325; A61K 31/39; A61K 31/405; C07C 251/24; C07C 255/43; C07C 271/16; C07D 327/04; C07D 411/12

(57) ABSTRACT

The present invention relates to compounds according to formula (I), which are prodrugs of the psychoactive compound phenethylamine or its derivatives. The prodrugs provided herein exhibit improved pharmacokinetic properties during uptake as compared to phenethylamine (or the respective phenethylamine derivative), as well as reduced side effects resulting from the metabolites thus formed. Due to the affinity of the active phenethylamine compound, inter alia, for the 5-HT₂ₐ-receptor, these prodrugs are particularly advantageous for use in therapy, e.g., in the treatment of depression, posttraumatic stress disorder (PTSD), Alzheimer's disease or dementia.

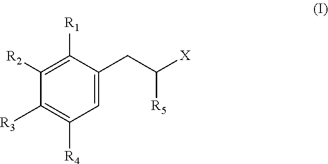

The present invention relates to compounds according to formula (I), which are prodrugs of the psychoactive compound phenethylamine or its derivatives. The prodrugs provided herein exhibit improved pharmacokinetic properties during uptake as compared to phenethylamine (or the respective phenethylamine derivative), as well as reduced side effects resulting from the metabolites thus formed. Due to the affinity of the active phenethylamine compound, inter alia, for the 5-HT₂ₐ-receptor, these prodrugs are particularly advantageous for use in therapy, e.g., in the treatment of depression or posttraumatic stress disorder (PTSD).

The ubiquitous family of phenethylamine compounds are represented by compounds like phenethylamine (PEA) and mescaline which are naturally occurring in cocoa beans and several species of cacti respectively. These compounds lead to various sympathomimetic effects inside the human body. In case of phenethylamine (PEA) the central nervous system is subjected to amphetamine-like stimulation. Due to the fact that phenethylamine (PEA) acts as a perfect substrate/inhibitor for the enzyme monoamine oxidase B (MAO B), these stimulative effects have, however, only a limited impact. Accordingly, the half-life of phenethylamine (PEA) after oral consumption is limited to approximately 10 minutes. Mescaline, nowadays studied for its potential against posttraumatic stress disorder (PTSD) and alcohol/drug abuse disorders, needs to be dosed orally at over 200 mg in order to cause the desired psychedelic state in patients. By the large dosage required by the metabolic characteristics of this agent, this compound is associated with side effects like nausea and hypertension. These effects make treatment complicated for sensitive patients or, even more severely, exclude a major group of patients from this therapy. To overcome or mitigate this issue of metabolic inactivation, numerous analogues of mescaline, like escaline or the 2C-X series, have been synthesized. The key modification of this series is limited to different substituents on the aromatic system of the compounds. Following a different strategy, modifications on the nitrogen led to new compounds with prodrug properties, including amfetaminil, fenproporex and fenetylline. By this molecular alternation, efficient prodrugs derived from amphetamine have been created to treat narcolepsy and attention deficit hyperactivity disorder (ADHD). In the case of fenethylline, the prodrug characteristics led to less side effects (compared to amphetamine) like increased blood pressure, enabling even the treatment of patients with cardiovascular conditions.

Molecular alterations mitigating these side effects while preserving the key characteristics of these compounds would be highly interesting for better tolerated therapies.

Novel active compounds, in particular those showing a modified (accelerated or retarded) activity in the human body due to their structure and consequently having a more favorable side-effect profile, are thus desired. Another important property of the thus designed “prodrugs” is a significantly decreased potential for abuse because these compounds cannot lead to a rapid “flooding” of the psychoactive phenethylamine in the human organism even at intravenous or intranasal application, and thus such compounds trigger dependencies to a lesser extent. In the context of the present invention, novel phenethylamine prodrugs based on imine- and carbamate-structures have been developed, which address the above-discussed issues.

Accordingly, in a first embodiment, the present invention relates to a compound of formula (I):

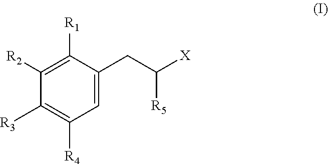

or a pharmaceutically acceptable salt thereof.

In formula (I), R₁ is selected from hydrogen, methyl and methoxy.
R₂ is selected from hydrogen, trifluoromethyl, methoxy and ethoxy, and
R₃ is selected from hydrogen, halogen, nitro, methyl, ethyl, n-propyl, trifluoromethyl, methoxy, ethoxy, n-propoxy, allyloxy, 2-methylallyloxy, ethylthio, n-propylthio, isopropylthio, cyclopropylmethylthio, tertbutylthio, and 2-fluoroethylthio; or
R₂ and R₃ together with the carbon atoms that carry R₂ and R₃, respectively, form a five membered ring selected from

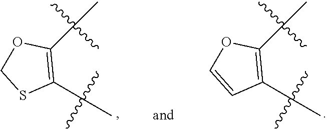 [SMILES: *C1=C(*)SCO1 ; *c1ccoc1*]

R₄ is selected from hydrogen, methyl and methoxy.
R₅ is selected from hydrogen and methyl.
X is selected from:

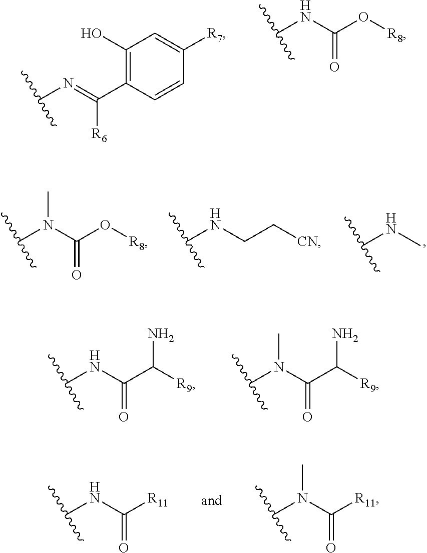

wherein R₆ is selected from C₁₋₅ alkyl and aryl, wherein said aryl is optionally substituted with one or more groups R^{S},
wherein R₇ is selected from methoxy and hydrogen,
wherein R₈ is C₁₋₅ alkyl,
wherein R₉ is selected from hydrogen, C₁₋₈ alkyl, C₂₋₈ alkenyl, C₂₋₈ alkynyl, —(C₁₋₆ alkylene)-O—R₁₀, —(C₁₋₆ alkylene)-S—R₁₀, —(C₁₋₆ alkylene)-N(R₁₀)—R₁₀, —(C₁₋₆ alkylene)-CO—R₁₀, —(C₁₋₆ alkylene)-COO—R₁₀, —(C₁₋₆ alkylene)-O—CO—(C₁₋₆ alkyl), —(C₁₋₆ alkylene)-CO—N(R₁₀)—R₁₀, —(C₁₋₆ alkylene)-N(R₁₀)—CO—(C₁₋₆ alkyl), —(C₁₋₆ alkylene)-CO—N(R₁₀)—O—R₁₀, —(C₁₋₆ alkylene)-O—CO—N(R₁₀)—R₁₀, —(C₁₋₆ alkylene)-N(R₁₀)—CO—N(R₁₀)—R₁₀, —(C₁₋₆ alkylene)-N(R₁₀)—C(═N—R₁₀)—N(R₁₀)—R₁₀, —(C₁₋₆ alkylene)-SO₃—R₁₀, —(C₁₋₆ alkylene)-carbocyclyl, and —(C₁₋₆ alkylene)-heterocyclyl, wherein the carbocyclyl group in said —(C₁₋₆ alkylene)-carbocyclyl and the heterocyclyl group in said —(C₁₋₆ alkylene)-heterocyclyl are each optionally substituted with one or more groups R^{S}, wherein said alkyl, said alkenyl, said alkynyl, and any alkylene group comprised in any of the aforementioned R₉ groups are each optionally substituted with one or more —OH, and further wherein each R₁₀ is independently selected from hydrogen and C₁₋₆ alkyl,
wherein R₁₁ is a C₁₋₈ alkyl, C₂₋₈ alkenyl, C₂₋₈ alkynyl, carbocyclyl or heterocyclyl, wherein said alkyl, said alkenyl, said alkynyl, said carbocyclyl and said heterocyclyl are each optionally substituted with one or more R^{S}, and
wherein each R^{S} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—CO—O—(C₁₋₅ alkyl), —(C₀₋₃ alkylene)-N(C₁₋₅ alkyl)-CO—O—(C₁₋₅ alkyl), —(C₀₋₃ alkylene)-O—CO—NH—(C₁₋₅ alkyl), —(C₀₋₃ alkylene)-O—CO—N(C₁₋₅ alkyl)-(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO₂—(C₁₋₅ alkyl), —(C₀₋₃ alkylene)-SO—(C₁₋₅ alkyl), —(C₀₋₃ alkylene)-carbocyclyl, and —(C₀₋₃ alkylene)-heterocyclyl, wherein the carbocyclyl moiety in said —(C₀₋₃ alkylene)-carbocyclyl and the heterocyclyl moiety in said —(C₀₋₃ alkylene)-heterocyclyl are each optionally substituted with one or more groups independently selected from C₁₋₄ alkyl, halogen, —CN, —NO₂, —OH, —O—(C₁₋₄ alkyl), —SH, —S—(C₁₋₄ alkyl), —NH₂, —NH(C₁₋₄ alkyl), —N(C₁₋₄ alkyl)(C₁₋₄ alkyl), —COOH, —COO(C₁₋₄ alkyl), —CONH₂, —CONH(C₁₋₄alkyl), —CON(C₁₋₄alkyl)(C₁₋₄alkyl), —NHCO(C₁₋₄alkyl) and —N(C₁₋₄alkyl)-CO(C₁₋₄alkyl).

In formula (I), if X is

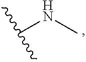 [SMILES: *NC]

then R₁ is hydrogen, R₂ and R₃ together with the carbon atoms that carry R₂ and R₃, respectively, form a five membered ring being

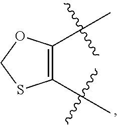 [SMILES: *C1=C(*)SCO1]

R₄ is hydrogen, and R₅ is methyl.

Moreover, if X is

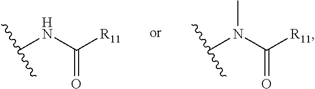

and if R₁₁ is a C₁₋₈ alkyl, C₂₋₈ alkenyl, C₂₋₈ alkynyl, or carbocyclyl, wherein said alkyl, said alkenyl, said alkynyl and said carbocyclyl are each optionally substituted with one or more R^{S}, then R₁ is hydrogen, R₂ is methoxy, R₃ is methoxy, R₄ is methoxy, and R₅ is hydrogen.

In a further embodiment, the present invention relates to a pharmaceutical composition comprising a compound of formula (I) or its pharmaceutically acceptable salt, and a pharmaceutically acceptable excipient.

In a further embodiment, the present invention relates to a compound of formula (I) or its pharmaceutically acceptable salt, or the pharmaceutical composition of the present invention, for use as a medicament.

In a further embodiment, the present invention relates to a compound of formula (I) or its pharmaceutically acceptable salt, or the pharmaceutical composition of the present invention, for use in the treatment of a serotonin 5-HT_{2A} receptor associated disease/disorder.

In a further embodiment, the present invention relates to use of the compound of formula (I) or its pharmaceutically acceptable salt in the manufacture of a medicament for the treatment of a serotonin 5-HT_{2A} receptor associated disease/disorder.

In a further embodiment, the present invention relates to a method of treating a serotonin 5-HT_{2A} receptor associated disease/disorder in a subject in need thereof, the method comprising administering a compound of formula (I) or its pharmaceutically acceptable salt, or the pharmaceutical composition of the present invention, to a subject in need thereof. It is to be understood that a therapeutically effective amount of the compound of formula (I) or its pharmaceutically acceptable salt, or the pharmaceutical composition of the present invention, is to be administered in accordance with the method.

The present invention is illustrated by the appended figures.

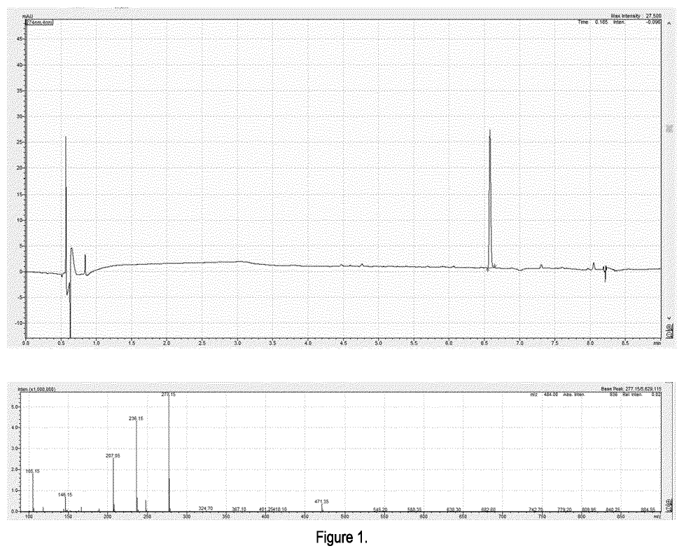

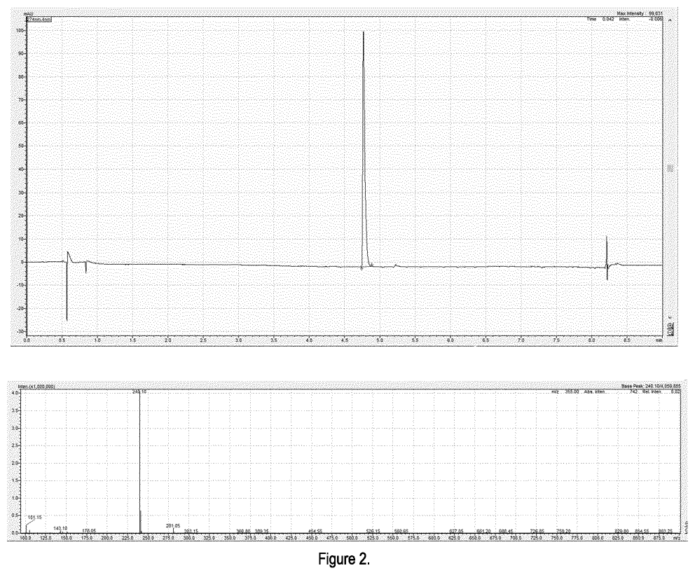

FIG. 1: LC-MS data obtained for the Preparative Example 1

FIG. 2: LC-MS data obtained for the Preparative Example 2.

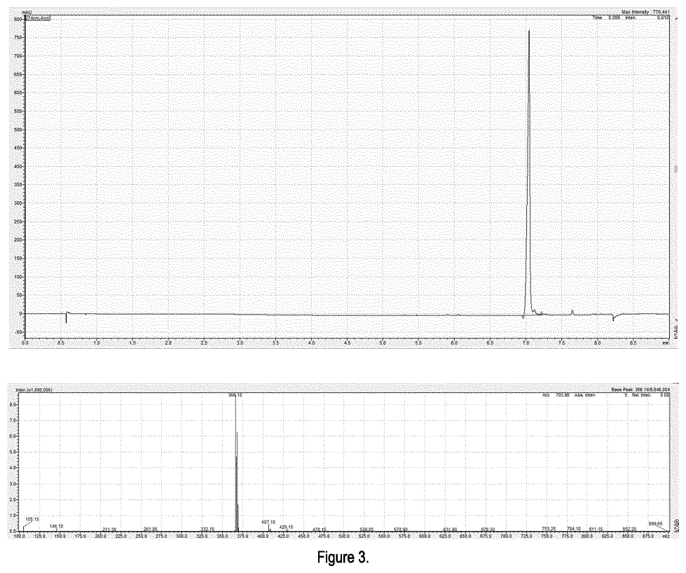

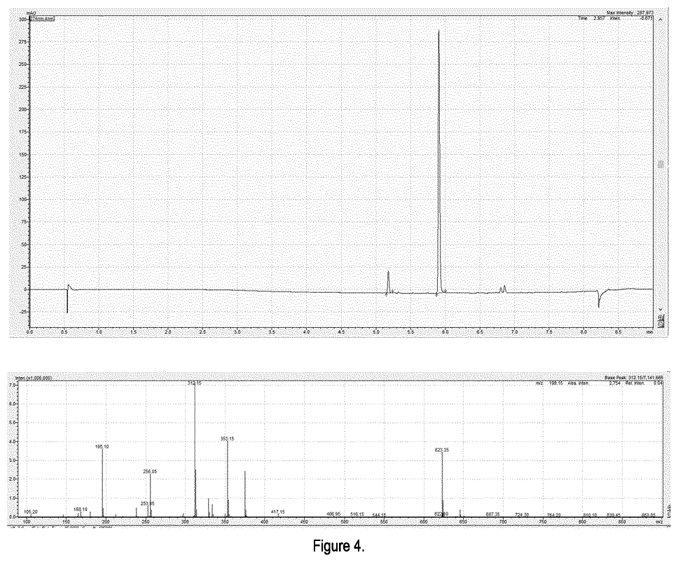

FIG. 3: LC-MS data obtained for the Preparative Example 3.

FIG. 4: LC-MS data obtained for the Preparative Example 4.

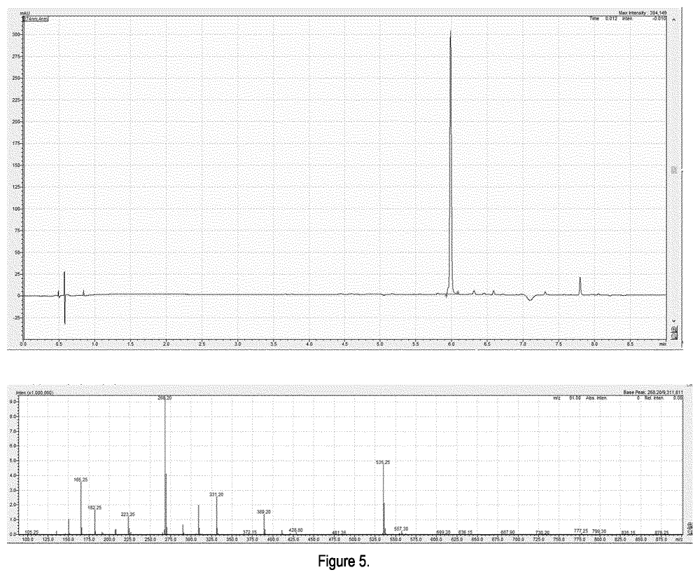

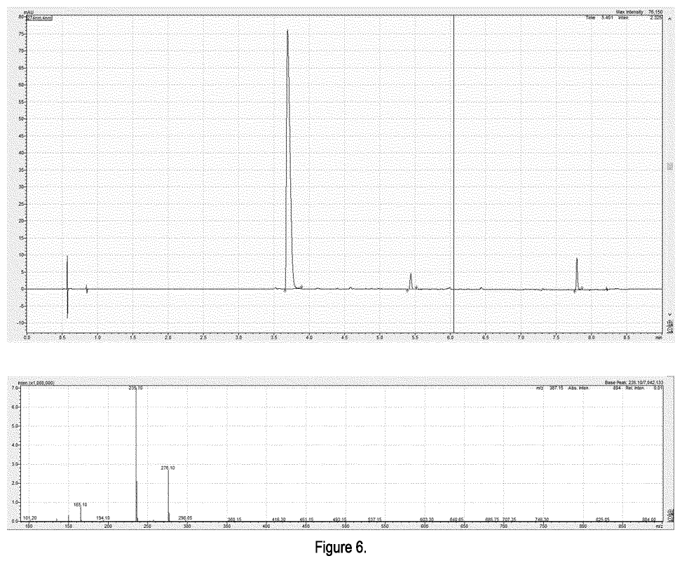

FIG. 5: LC-MS data obtained for the Preparative Example 5.

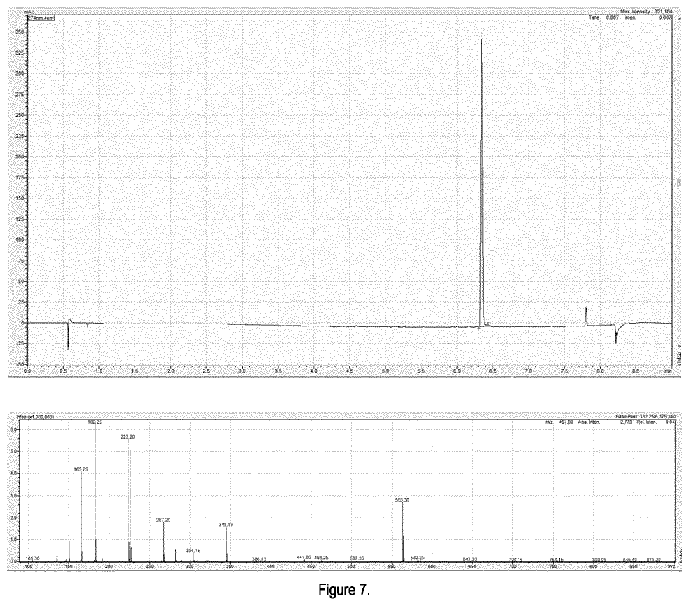

FIG. 6: LC-MS data obtained for the Preparative Example 6.

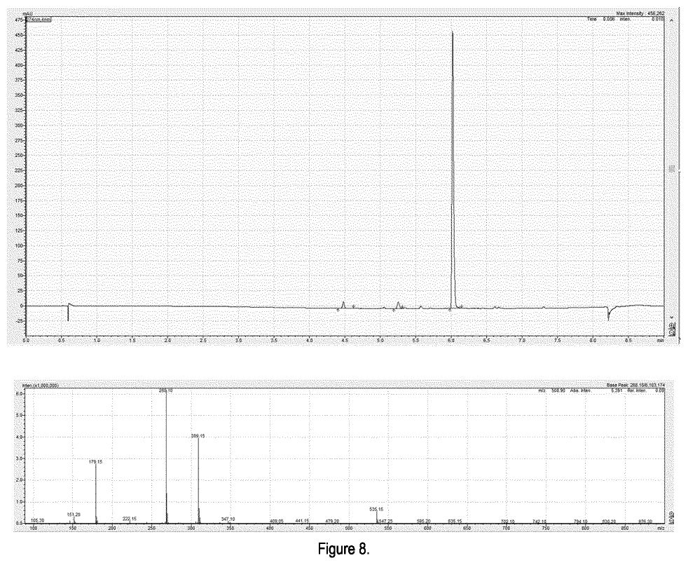

FIG. 7: LC-MS data obtained for the Preparative Example 7.

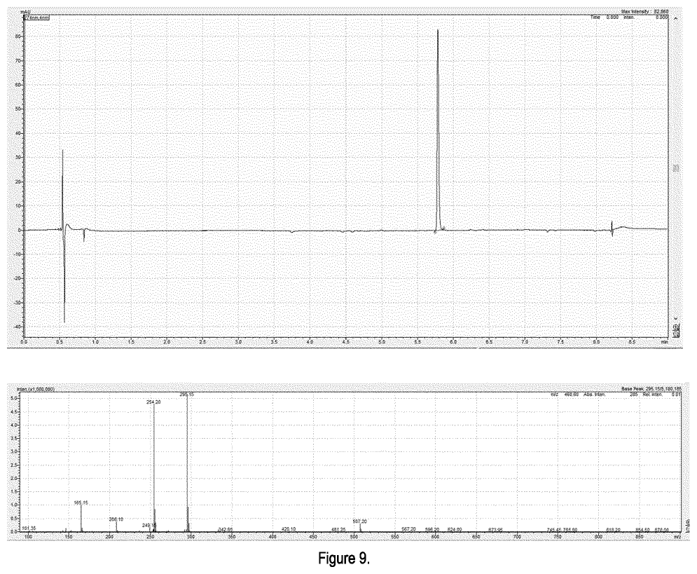

FIG. 8: LC-MS data obtained for the Preparative Example 8.

FIG. 9: LC-MS data obtained for the Preparative Example 9.

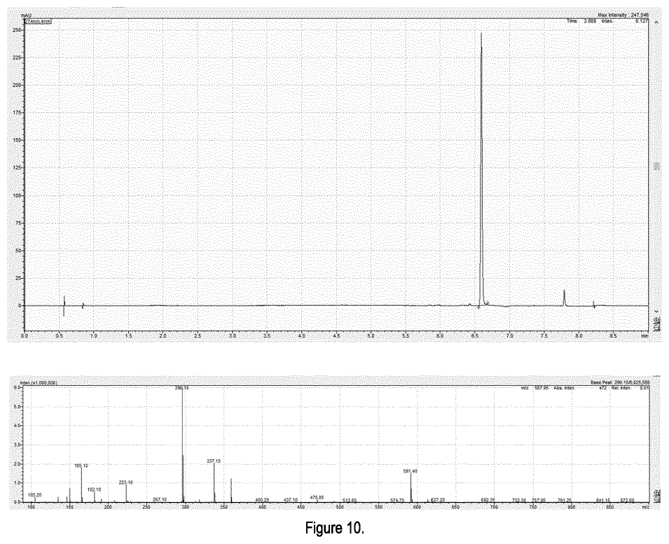

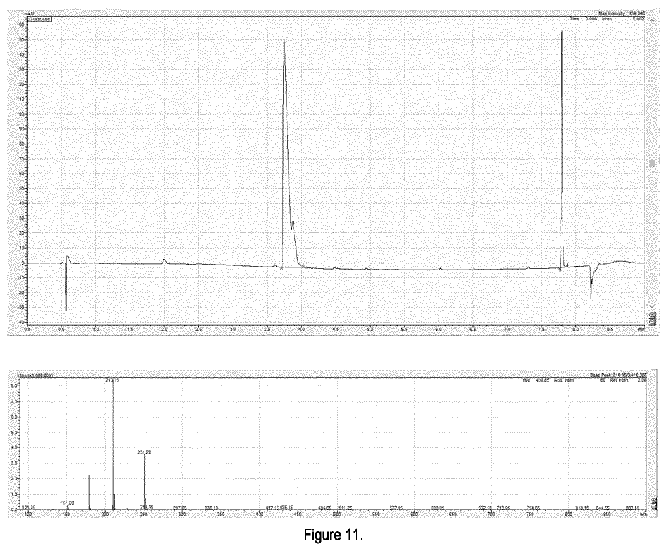

FIG. 10: LC-MS data obtained for the Preparative Example 10.

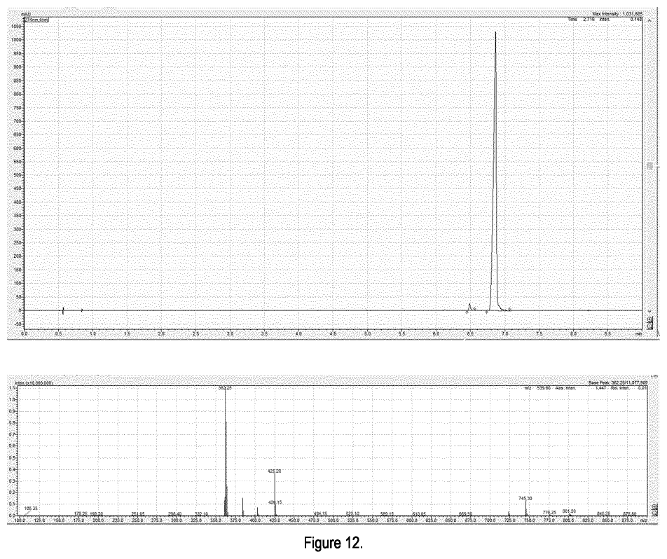

FIG. 11: LC-MS data obtained for the Preparative Example 11.

FIG. 12: LC-MS data obtained for the Preparative Example 12.

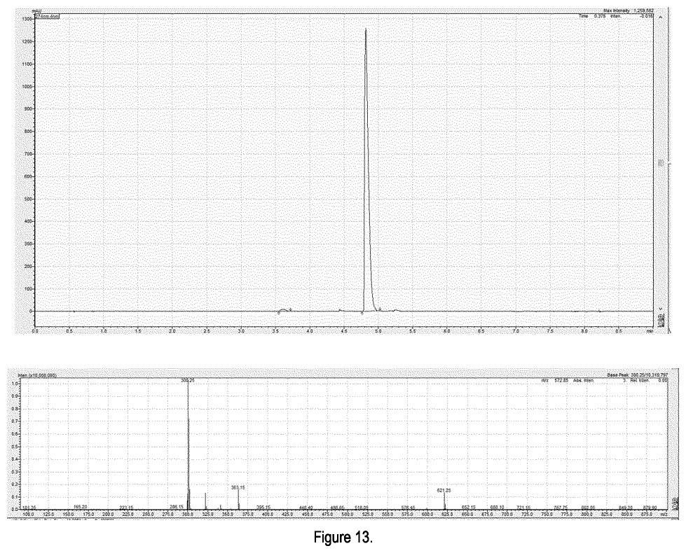

FIG. 13: LC-MS data obtained for the Preparative Example 14.

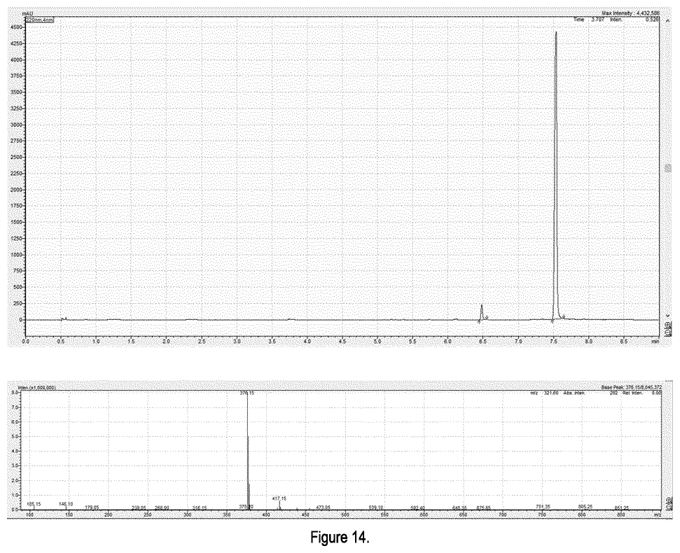

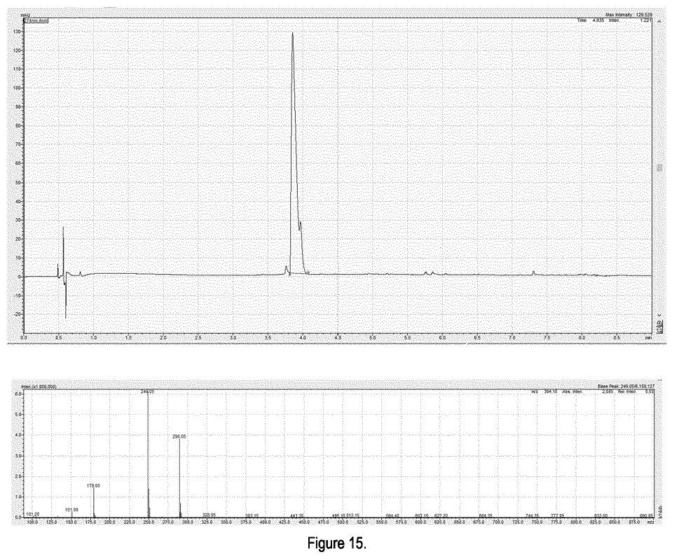

FIG. 14: LC-MS data obtained for the Preparative Example 15.

FIG. 15: LC-MS data obtained for the Preparative Example 16.

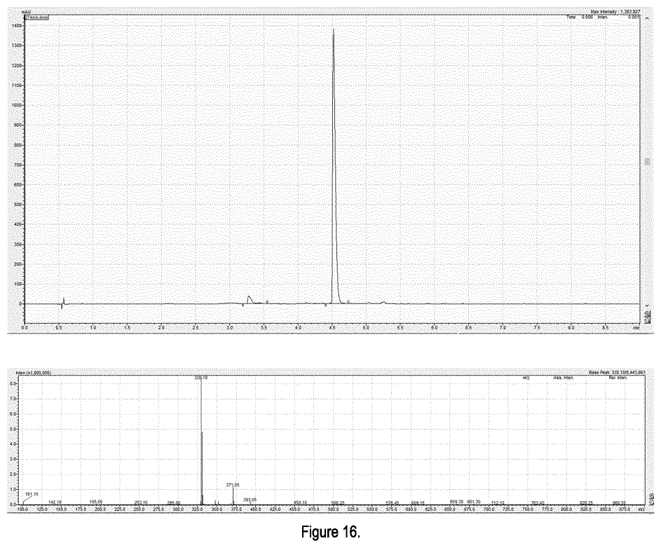

FIG. 16: LC-MS data obtained for the Preparative Example 17.

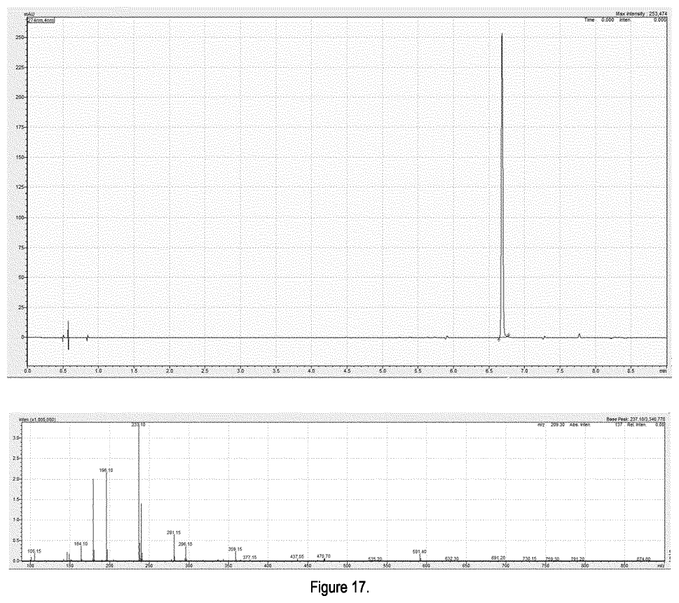

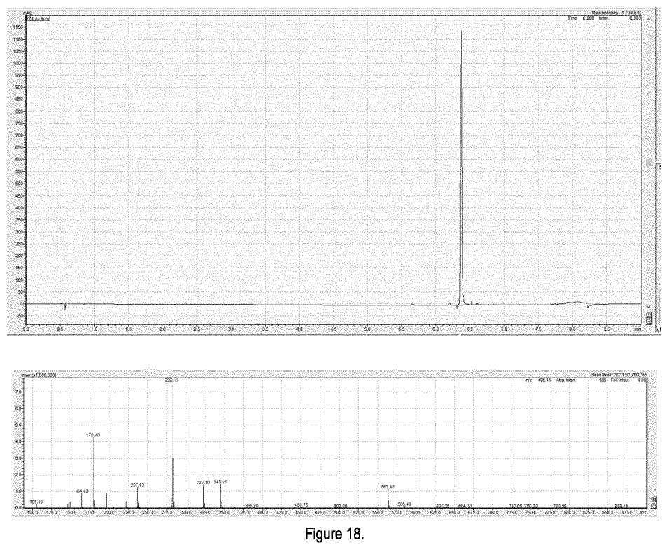

FIG. 17: LC-MS data obtained for the Preparative Example 18.

FIG. 18: LC-MS data obtained for the Preparative Example 19.

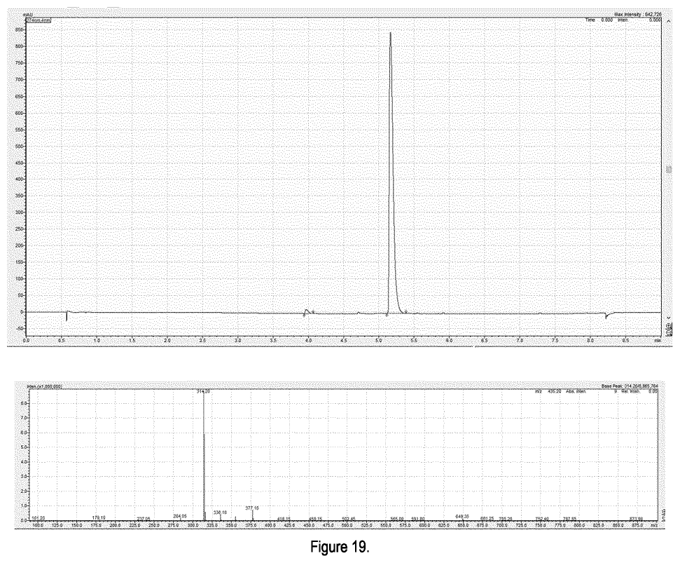

FIG. 19: LC-MS data obtained for the Preparative Example 20.

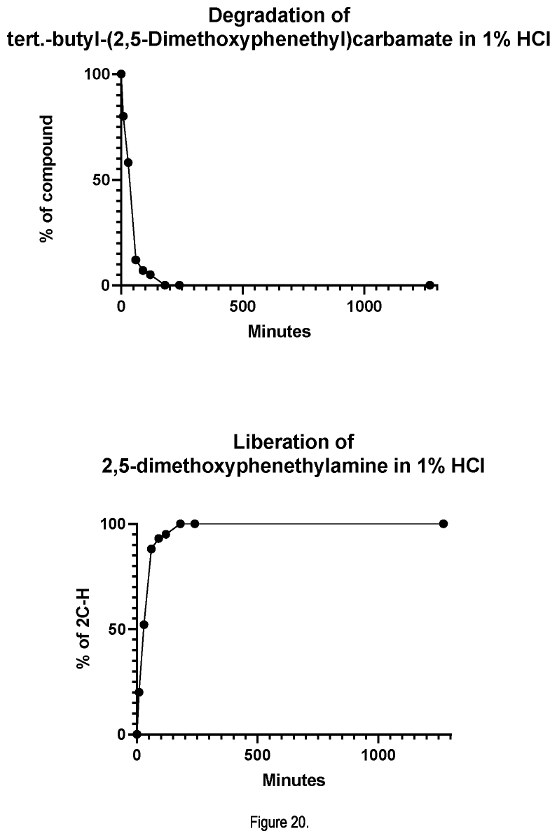

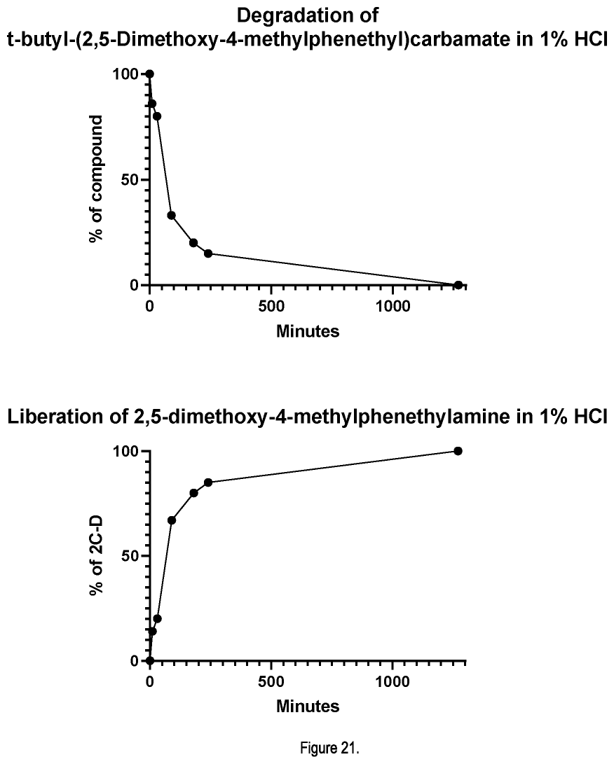

FIG. 20: Study of the decomposition of tert-butyl-(2,5-dimethoxyphenethyl) carbamate—the compound according to Preparative Example 7 (upper) and formation of 2,5-dimethoxyphenethylamine (lower) in 1% HCl (representative of gastric acid present within stomach).

FIG. 21: Study of the decomposition of tert-butyl (2,5-dimethoxy-4-methylphenethyl) carbamate—the compound according to Preparative Example 18 (upper) and formation of 2,5-dimethoxy-4-methylphenetyhlamine (lower) in 1% HCl (representative of gastric acid present within stomach).

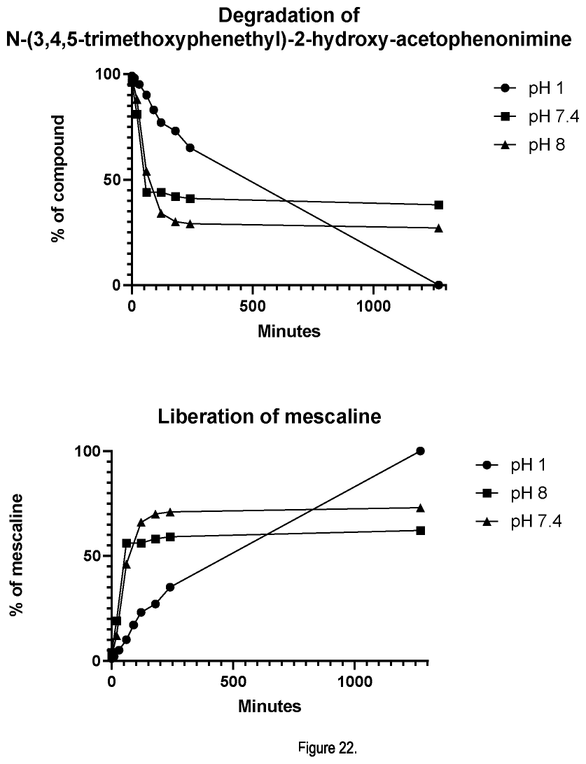

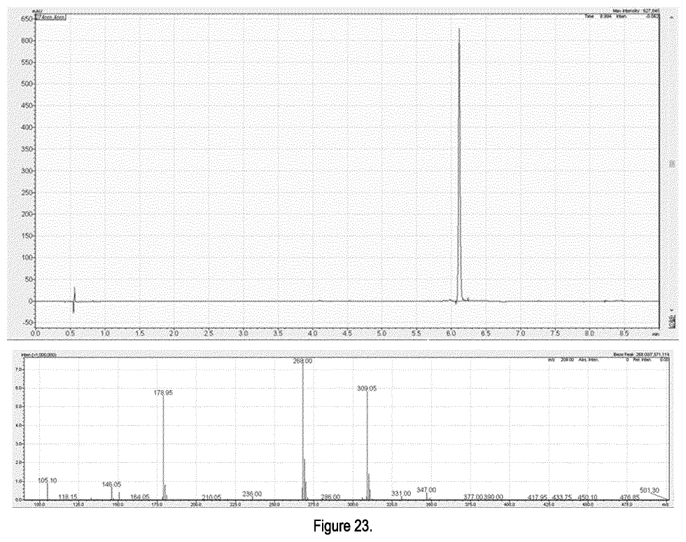

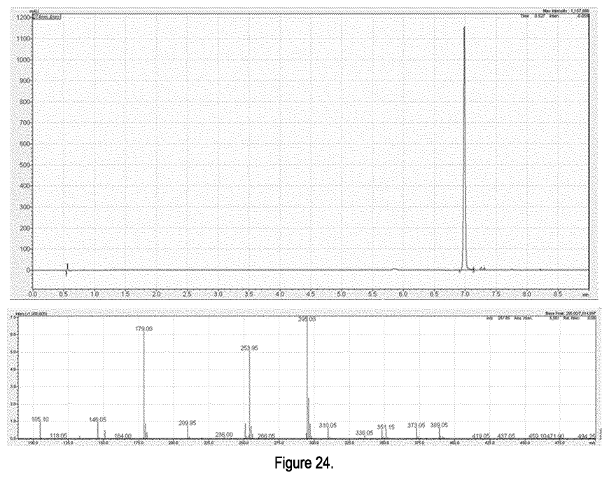

FIG. 22: Study of the decomposition of N-(3,4,5-trimethoxyphenethyl)-2-hydroxy-acetophenonimine—the compound according to Preparative Example 17 (upper) and formation of mescaline (lower) at pH=1, at pH=7.4 and at pH=8.

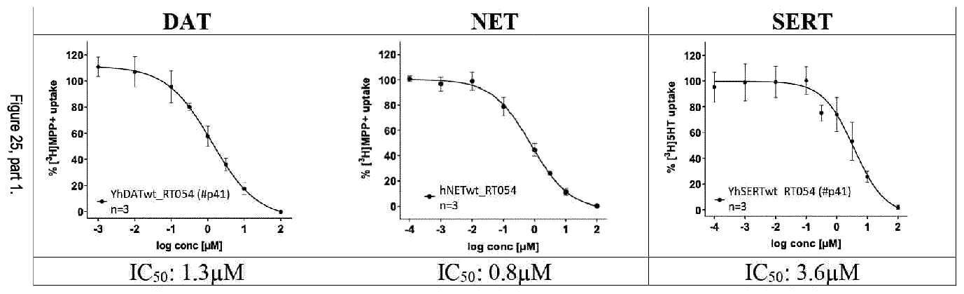

FIG. 23: LC-MS data obtained for the Preparative Example 21.

FIG. 24: LC-MS data obtained for the Preparative Example 22.

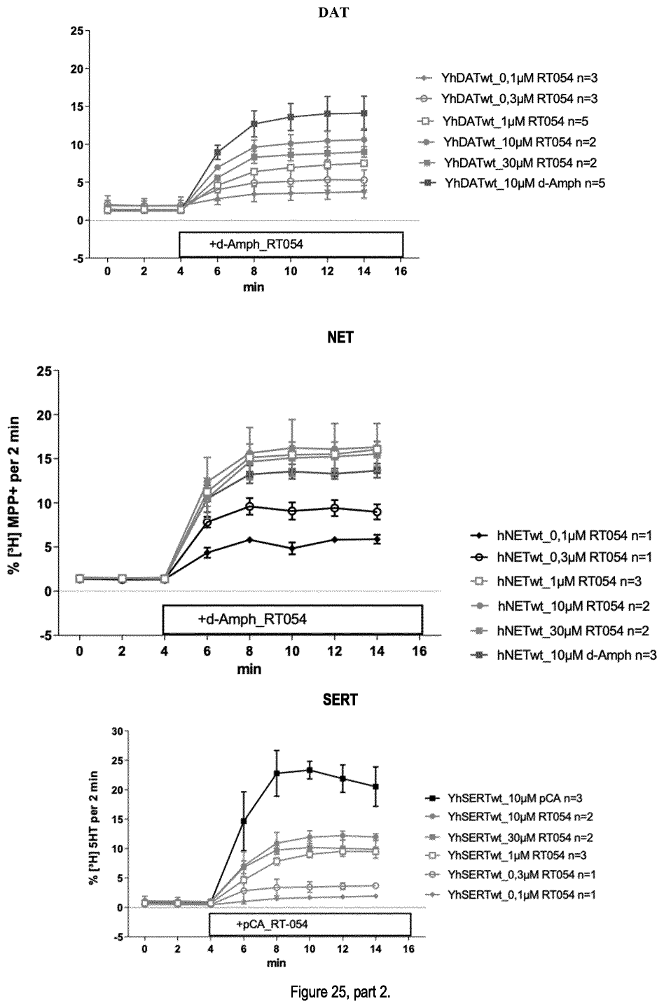

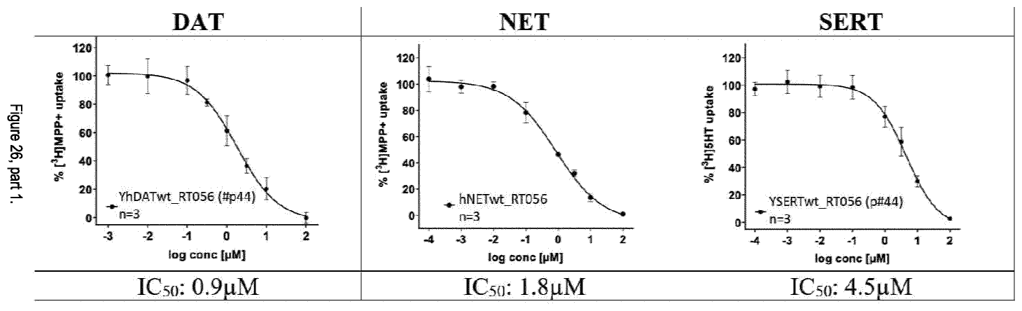

FIG. 25: Part 1: Uptake inhibition assays for transporters DAT, NET and SERT done with compound RT54; Part 2: Release assays for transporters DAT. NET and SERT done with compound RT54.

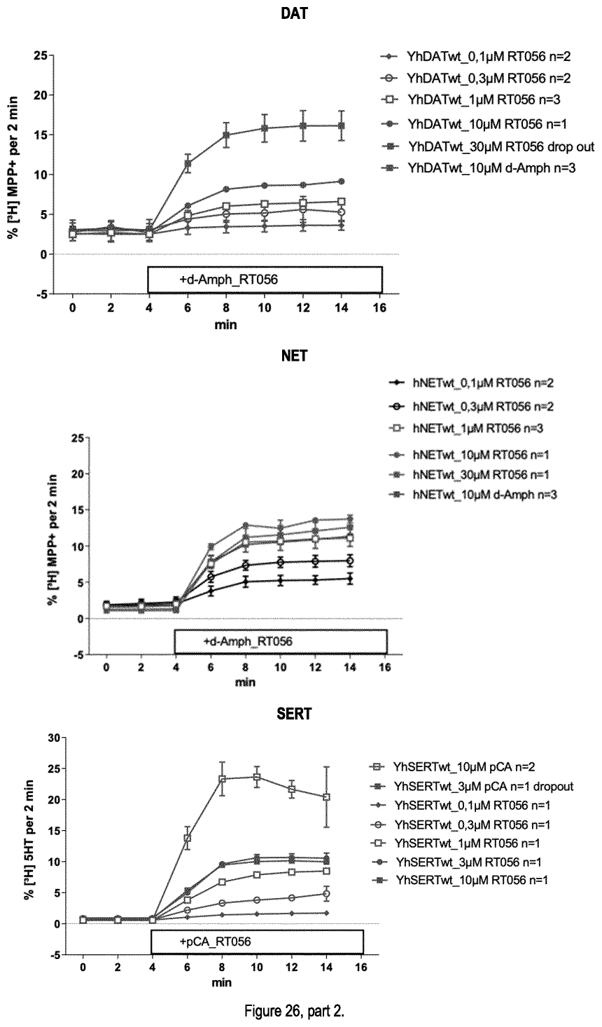

FIG. 26: Part 1: Uptake inhibition assays for transporters DAT, NET and SERT done with compound RT56; Part 2: Release assays for transporters DAT. NET and SERT done with compound RT56.

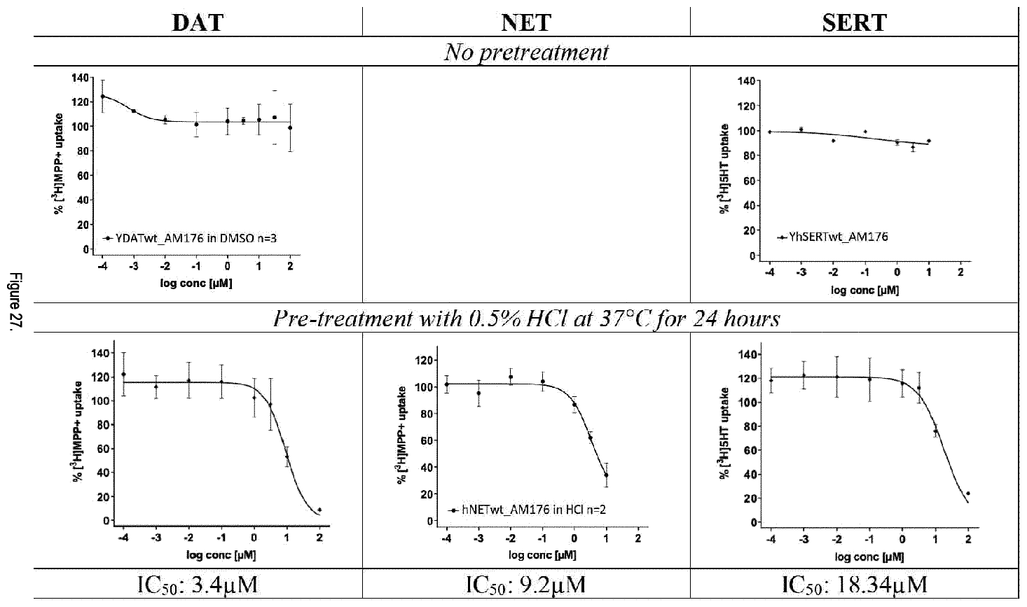

FIG. 27: Uptake inhibition assays for transporters DAT, NET and SERT done with compound AM176 in the absence of and with pretreatment with 0.5% HCl at 37° C. for 24 hours.

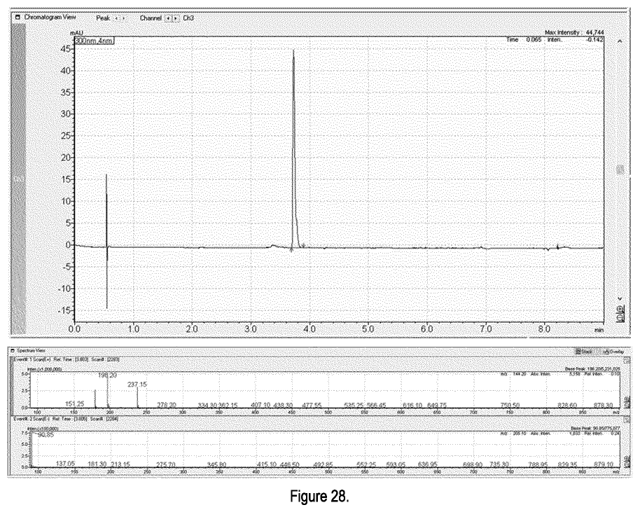

FIG. 28 LC-MS data obtained for the Preparative Example 26.

As explained above, the present invention relates to a compound of formula (I)

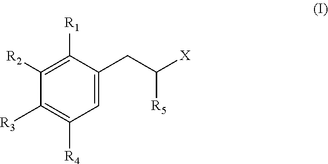

or a pharmaceutically acceptable salt thereof.

The following detailed description of the compound of formula (I) relates to all embodiments of the present invention, and is also applicable to all salts (in particular pharmaceutically acceptable salts) of the compound of formula (I).
R₁ is selected from hydrogen, methyl and methoxy. Preferably, R₁ is selected from hydrogen and methoxy.
R₂ is selected from hydrogen, trifluoromethyl, methoxy and ethoxy, and
R₃ is selected from hydrogen, halogen, nitro, methyl, ethyl, n-propyl, trifluoromethyl, methoxy, ethoxy, n-propoxy, allyloxy, 2-methylallyloxy, ethylthio, n-propylthio, isopropylthio, cyclopropylmethylthio, tertbutylthio, and 2-fluoroethylthio; or
R₂ and R₃ together with the carbon atoms that carry R₂ and R₃, respectively, form a five membered ring selected from

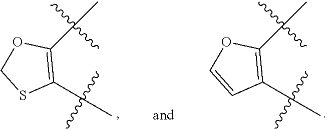 [SMILES: *C1=C(*)SCO1 ; *c1ccoc1*]

It will be understood that if R₂ and R₃ together with the carbon atoms that carry R₂ and R₃, respectively, form a five membered ring, then the corresponding five membered ring is fused/condensed to the phenyl ring carrying R₂ and R₃, so that the resulting compound has a fused bicyclic group.

If R₂ and R₃, together with the carbon atoms that carry R₂ and R₃, form a five membered ring, it is preferred that said five membered ring is

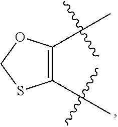 [SMILES: *C1=C(*)SCO1]

and more preferably said five membered ring is

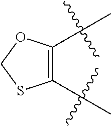 [SMILES: *C1=C(*)SCO1]

wherein the carbon atom carrying R₃ is connected to the S atom in said five membered ring

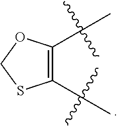 [SMILES: *C1=C(*)SCO1]

Notwithstanding the above description of preferred groups in the case that R₂ and R₃, together with the carbon atoms carrying R₂ and R₃, form a five membered ring, it is preferred that R₂ and R₃ do not form a five membered ring.

Accordingly, it is preferred that R₂ is selected from hydrogen, trifluoromethyl, methoxy and ethoxy, and
R₃ is selected from hydrogen, halogen, nitro, methyl, ethyl, propyl, trifluoromethyl, methoxy, ethoxy, n-propoxy, allyloxy, 2-methylallyloxy, ethylthio, n-propylthio, isopropylthio, cyclopropylmethylthio, tertbutylthio, and 2-fluoroethylthio.

More preferably, R₂ is selected from hydrogen and methoxy, and R₃ is selected from hydrogen, halogen, methyl, ethyl, propyl, trifluoromethyl, methoxy, and ethoxy.

R₄ is selected from hydrogen, methyl and methoxy.

Preferably, R₄ is selected from hydrogen and methoxy.

R₅ is selected from hydrogen and methyl.

X is selected from:

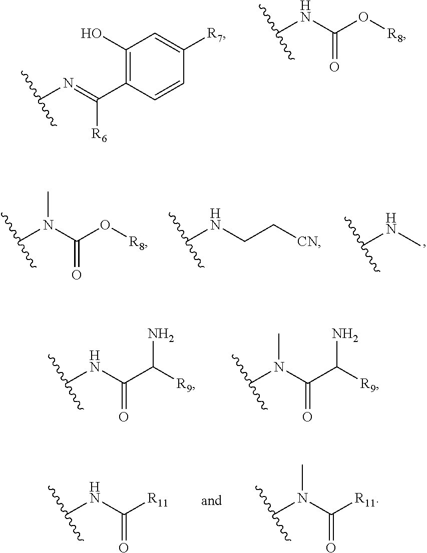

Preferably, X is selected from:

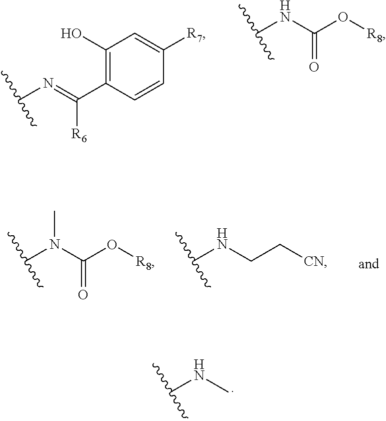

In particular, X may be selected from:

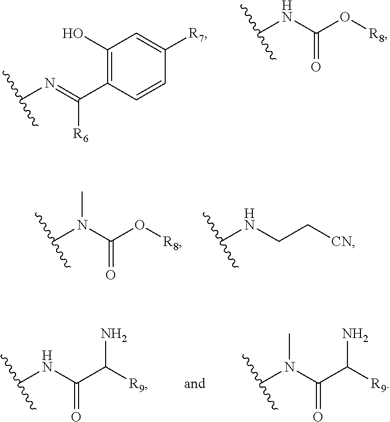

R₆ is selected from C₁₋₅ alkyl and aryl, wherein said aryl is optionally substituted with one or more groups R^{S}. Exemplary C₁₋₅ alkyl groups include methyl, ethyl or n-propyl. A particularly preferred C₁₋₅ alkyl is methyl. A particularly preferred aryl group is phenyl, wherein said phenyl is optionally substituted with one or more groups R^{S}. Thus preferably, R₆ is selected from methyl and phenyl wherein said phenyl is optionally substituted with one or more groups R^{S}. More preferably, R₆ is selected from methyl, phenyl and 4-chloro-phenyl.

R₇ is selected from methoxy and hydrogen.

R₈ is C₁₋₅ alkyl. Exemplary C₁₋₅ alkyl groups include ethyl, isopropyl, isobutyl, tert-butyl or neopentyl. Thus preferably, R₈ is selected from ethyl, isopropyl, isobutyl, tert-butyl and neopentyl.

R₉ is selected from hydrogen, C₁₋₈ alkyl, C₂₋₈ alkenyl, C₂₋₈ alkynyl, —(C₁₋₆ alkylene)-O—R₁₀, —(C₁₋₆ alkylene)-S—R₁₀, —(C₁₋₆ alkylene)-N(R₁₀)—R₁₀, —(C₁₋₆ alkylene)-CO—R₁₀, —(C₁₋₆ alkylene)-COO—R₁₀, —(C₁₋₆ alkylene)-O—CO—(C₁₋₆ alkyl), —(C₁₋₆ alkylene)-CO—N(R₁₀)—R₁₀, —(C₁₋₆ alkylene)-N(R₁₀)—CO—(C₁₋₆ alkyl), —(C₁₋₆ alkylene)-CO—N(R₁₀)—O—R₁₀, —(C₁₋₆ alkylene)-O—CO—N(R₁₀)—R₁₀, —(C₁₋₆ alkylene)-N(R₁₀)—CO—N(R₁₀)—R₁₀, —(C₁₋₆ alkylene)-N(R₁₀)—C(═N—R₁₀)—N(R₁₀)—R₁₀, —(C₁₋₆ alkylene)-SO₃—R₁₀, —(C₁₋₆ alkylene)-carbocyclyl, and —(C₀₋₆ alkylene)-heterocyclyl, wherein the carbocyclyl group in said —(C₁₋₆ alkylene)-carbocyclyl and the heterocyclyl group in said —(C₁₋₆ alkylene)-heterocyclyl are each optionally substituted with one or more groups R^{S}, wherein said alkyl, said alkenyl, said alkynyl, and any alkylene group comprised in any of the aforementioned R₉ groups are each optionally substituted with one or more —OH, and further wherein each R₁₀ is independently selected from hydrogen and C₁₋₆ alkyl.

Preferably, R₉ is selected from hydrogen, C₁₋₈ alkyl, C₂₋₈ alkenyl, C₂₋₈ alkynyl, —(C₁₋₆ alkylene)-OH, —(C₁₋₆ alkylene)-O(C₁₋₆ alkyl), —(C₁₋₆ alkylene)-SH, —(C₁₋₆ alkylene)-S(C₁₋₆ alkyl), —(C₁₋₆ alkylene)-NH₂, —(C₁₋₆ alkylene)-NH—CO—NH₂, —(C₁₋₆ alkylene)-NH—C(═NH)—NH₂, —(C₁₋₆ alkylene)-O—CO—NH₂, —(C₁₋₆ alkylene)-COOH, —(C₁₋₆ alkylene)-CO—NH₂, —(C₁₋₆ alkylene)-CO—NH—OH, —(C₁₋₆ alkylene)-SO₃H, phenyl, —(C₁₋₆ alkylene)-phenyl, cycloalkyl, —(C₁₋₆ alkylene)-cycloalkyl, heteroaryl, —(C₁₋₆ alkylene)-heteroaryl, heterocycloalkyl, and —(C₁₋₆ alkylene)-heterocycloalkyl, wherein said phenyl, the phenyl group in said —(C₁₋₆ alkylene)-phenyl, said cycloalkyl, the cycloalkyl group in said —(C₁₋₆ alkylene)-cycloalkyl, said heteroaryl, the heteroaryl group in said —(C₁₋₆ alkylene)-heteroaryl, said heterocycloalkyl, and the heterocycloalkyl group in said —(C₁₋₆ alkylene)-heterocycloalkyl are each optionally substituted with one or more groups R^{S}, and further wherein said alkyl, said alkenyl, said alkynyl, and any alkylene group in any of the aforementioned groups are each optionally substituted with one or more —OH.

More preferably, R₉ is selected from hydrogen, C₁₋₈ alkyl, C₂₋₈ alkenyl, C₂₋₈ alkynyl, phenyl, —(C₁₋₆ alkylene)-phenyl, cycloalkyl, —(C₁₋₆ alkylene)-cycloalkyl, heteroaryl, —(C₁₋₆ alkylene)-heteroaryl, heterocycloalkyl, and —(C₁₋₆ alkylene)-heterocycloalkyl, wherein said phenyl, the phenyl group in said —(C₁₋₆ alkylene)-phenyl, said cycloalkyl, the cycloalkyl group in said —(C₁₋₆ alkylene)-cycloalkyl, said heteroaryl, the heteroaryl group in said —(C₁₋₆ alkylene)-heteroaryl, said heterocycloalkyl, and the heterocycloalkyl group in said —(C₁₋₆ alkylene)-heterocycloalkyl are each optionally substituted with one or more groups R^{S}, and further wherein said alkyl, said alkenyl, said alkynyl, and any alkylene group in any of the aforementioned groups are each optionally substituted with one or more —OH.

Even more preferably, R₉ is selected from hydrogen, C₁₋₈ alkyl, —(C₁₋₆ alkylene)-phenyl, and —(C₁₋₆ alkylene)-heteroaryl.

Still more preferably, R₉ is selected from hydrogen, methyl, benzyl, and —CH₂-(indol-3-yl). Particularly preferred examples of R₉ include hydrogen or —CH₂-(1H-indol-3-yl).

R₁₁ is a C₁₋₈ alkyl, C₂₋₈ alkenyl, C₂₋₈ alkynyl, carbocyclyl or heterocyclyl, wherein said alkyl, said alkenyl, said alkynyl, said carbocyclyl and said heterocyclyl are each optionally substituted with one or more R^{S}. Preferably, R₁₁ is a heterocyclyl, wherein said heterocyclyl is optionally substituted with one or more R^{S}. More preferably, R₁₁ is a heterocyclyl, wherein said heterocyclyl is attached via a ring carbon atom that is directly adjacent to a ring nitrogen atom, and further wherein said heterocyclyl is optionally substituted with one or more groups R^{S}. Even more preferably, R₁₁ is a heterocyclyl, wherein said heterocyclyl is attached via a ring carbon atom that is directly adjacent to a ring nitrogen atom, wherein said heterocyclyl is optionally substituted with one or more (e.g., one, two, three, four, or five) R^{S}, and wherein said heterocyclyl is a heterocycloalkyl, a heterocycloalkenyl, or a heteroaryl (wherein said heterocycloalkyl, said heterocycloalkenyl, or said heteroaryl may each be monocyclic or polycyclic, e.g., bicyclic or tricyclic). Yet even more preferably, R₁₁ is pyrrolidin-2-yl, 4-hydroxypyrrolidin-2-yl, 5-oxo-pyrrolidin-2-yl, or piperidin-2-yl.

Each R^{S} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—CO—O—(C₁₋₅ alkyl), —(C₀₋₃ alkylene)-N(C₁₋₅ alkyl)-CO—O—(C₁₋₅ alkyl), —(C₀₋₃ alkylene)-O—CO—NH—(C₁₋₅ alkyl), —(C₀₋₃ alkylene)-O—CO—N(C₁₋₅ alkyl)-(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO₂—(C₁₋₅ alkyl), —(C₀₋₃ alkylene)-SO—(C₁₋₅ alkyl), —(C₀₋₃ alkylene)-carbocyclyl, and —(C₀₋₃ alkylene)-heterocyclyl, wherein the carbocyclyl moiety in said —(C₀₋₃ alkylene)-carbocyclyl and the heterocyclyl moiety in said —(C₀₋₃ alkylene)-heterocyclyl are each optionally substituted with one or more groups independently selected from C₁₋₄ alkyl, halogen, —CN, —NO₂, —OH, —O—(C₁₋₄ alkyl), —SH, —S—(C₁₋₄ alkyl), —NH₂, —NH(C₁₋₄ alkyl), —N(C₁₋₄ alkyl)(C₁₋₄ alkyl), —COOH, —COO(C₁₋₄ alkyl), —CONH₂, —CONH(C₁₋₄ alkyl), —CON(C₁₋₄ alkyl)(C₁₋₄ alkyl), —NHCO(C₁₋₄ alkyl) and —N(C₁₋₄ alkyl)-CO(C₁₋₄ alkyl).

Preferably, each R^{S} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—CO—O—(C₁₋₅ alkyl), —(C₀₋₃ alkylene)-N(C₁₋₅ alkyl)-CO—O—(C₁₋₅ alkyl), —(C₀₋₃ alkylene)-O—CO—NH—(C₁₋₅ alkyl), —(C₀₋₃ alkylene)-O—CO—N(C₁₋₅ alkyl)-(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO₂—(C₁₋₅ alkyl), and —(C₀₋₃ alkylene)-SO—(C₁₋₅ alkyl).

More preferably, each R^{S} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), and —(C₀₋₃ alkylene)-halogen.

Even more preferably, each R^{S} is independently selected from C₁₋₅ alkyl, C₂₋₅ alkenyl, C₂₋₅ alkynyl, —OH, —O(C₁₋₅ alkyl), —SH, —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), and halogen.

Even more preferably, each R^{S} is independently selected from —OH and halogen.

Yet even more preferably, each R^{S} is independently halogen. A particularly preferred halogen as R^{S} is chloro.

In the compound of formula (I), if X is

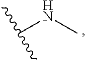 [SMILES: *NC]

then R₁ is hydrogen, R₂ and R₃ together with the carbon atoms that carry R₂ and R₃, respectively, form a five membered ring being

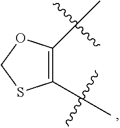 [SMILES: *C1=C(*)SCO1]

R₄ is hydrogen, and R₅ is methyl.

Preferably, the carbon atom carrying R₃ is connected to the S atom in said group

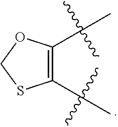 [SMILES: *C1=C(*)SCO1]

Moreover, in the compound of formula (I), if X is

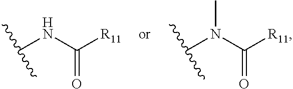

and if R₁₁ is a C₁₋₈ alkyl, C₂₋₈ alkenyl, C₂₋₈ alkynyl, or carbocyclyl, wherein said alkyl, said alkenyl, said alkynyl and said carbocyclyl are each optionally substituted with one or more R^{S}, then R₁ is hydrogen, R₂ is methoxy, R₃ is methoxy, R₄ is methoxy, and R₅ is hydrogen.

Preferably, in the compound of formula (I), if X is

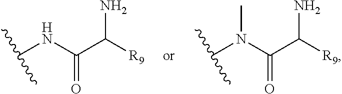

then R₁ is hydrogen, R₂ and R₃ together with the carbon atoms that carry R₂ and R₃, respectively, form a five membered ring being

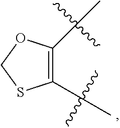 [SMILES: *C1=C(*)SCO1]

R₄ is hydrogen, and R₅ is methyl. Preferably, the carbon atom carrying R₃ is connected to the S atom in said group

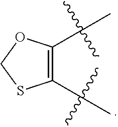 [SMILES: *C1=C(*)SCO1]

In the compound of formula (I), if X is

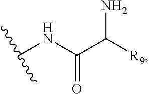

it is preferred that said group X is

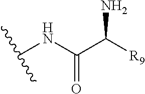

(i.e., it is preferred that said group X has the depicted specific configuration).

In the compound of formula (I), if X is

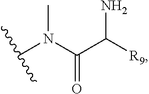

it is preferred that said group X is

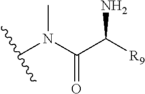

In the compound of formula (I), if X is

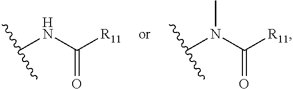

and if R₁₁ is pyrrolidin-2-yl, 4-hydroxypyrrolidin-2-yl or 5-oxo-pyrrolidin-2-yl, then it is preferred that said pyrrolidin-2-yl is

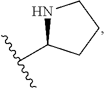 [SMILES: *[C@@H]1CCCN1]

said 4-hydroxypyrrolidin-2-yl is

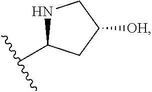 [SMILES: *[C@@H]1C[C@@H](O)CN1]

and said 5-oxo-pyrrolidin-2-yl is

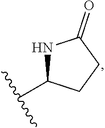 [SMILES: *[C@@H]1CCC(=O)N1]

respectively.

Preferably, in the compound of formula (I), as defined hereinabove, X is selected from:

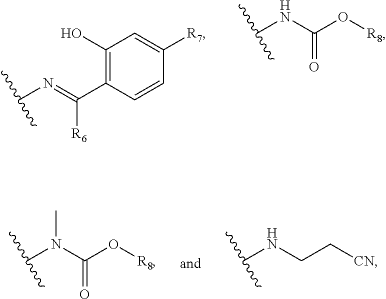

wherein R₆, R₇ and R₈ are as defined herein.

More preferably, in the compound of formula (I), as defined hereinabove, X is selected from:

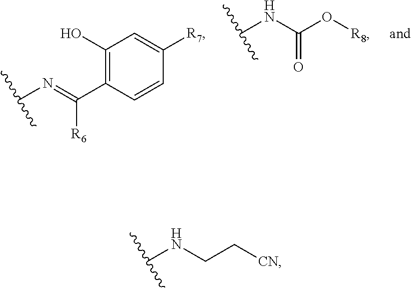

wherein R₆, R₇ and R₈ are as defined herein.

Even more preferably, in the compound of formula (I), as defined hereinabove, X is selected from:

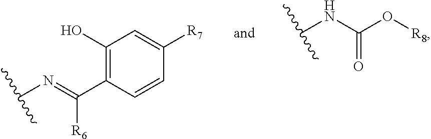

wherein R₆, R₇ and R₈ are as defined herein.

Particularly preferred compounds of formula (I) are the compounds wherein R₁, R₂, R₃, R₄, and R₅ are as defined in any one of the following specific embodiments, and wherein X is as defined hereinabove.

In a first specific embodiment of the compound of formula (I), R₁ is hydrogen, R₂ is methoxy, R₃ is methoxy, R₄ is methoxy, and R₅ is hydrogen.

In a second specific embodiment of the compound of formula (I), R₁ is hydrogen, R₂ is n-propoxy, R₃ is methoxy, R₄ is methoxy, and R₅ is hydrogen.

In a third specific embodiment of the compound of formula (I), R₁ is hydrogen, R₂ is ethoxy, R₃ is methoxy, R₄ is methoxy, and R₅ is hydrogen.

In a fourth specific embodiment of the compound of formula (I), R₁ is hydrogen, R₂ is methoxy, R₃ is 2-allyloxy, R₄ is methoxy, and R₅ is hydrogen.

In a fifth specific embodiment of the compound of formula (I), R₁ is hydrogen, R₂ is methoxy, R₃ is 2-methyl-2-allyloxy, R₄ is methoxy, and R₅ is hydrogen.

In a sixth specific embodiment of the compound of formula (I), R₁ is hydrogen, R₂ is ethoxy, R₃ is ethoxy, R₄ is methoxy, and R₅ is hydrogen.

In a seventh specific embodiment of the compound of formula (I), R₁ is hydrogen, R₂ is methoxy, R₃ is methyl, R₄ is methoxy, and R₅ is methyl.

In an eighth specific embodiment of the compound of formula (I), R₁ is hydrogen, R₂ is methoxy, R₃ is bromo, R₄ is methoxy, and R₅ is methyl.

In a ninth specific embodiment of the compound of formula (I), R₁ is hydrogen, R₂ is methoxy, R₃ is iodo, R₄ is methoxy, and R₅ is methyl.

In a tenth specific embodiment of the compound of formula (I), R₁ is hydrogen, R₂ is methoxy, R₃ is chloro, R₄ is methoxy, and R₅ is methyl.

In an eleventh specific embodiment of the compound of formula (I), R₁ is hydrogen, R₂ is methoxy, R₃ is nitro, R₄ is methoxy, and R₅ is methyl.

In a twelfth specific embodiment of the compound of formula (I), R₁ is hydrogen, R₂ is methoxy, R₃ is bromo, R₄ is methoxy, and R₅ is hydrogen.

In a thirteenth specific embodiment of the compound of formula (I), R₁ is hydrogen, R₂ is methoxy, R₃ is chloro, R₄ is methoxy, and R₅ is hydrogen.

In a fourteenth specific embodiment of the compound of formula (I), R₁ is hydrogen, R₂ is methoxy, R₃ is iodo, R₄ is methoxy, and R₅ is hydrogen.

In a fifteenth specific embodiment of the compound of formula (I), R₁ is hydrogen, R₂ is methoxy, R₃ is methyl, R₄ is methoxy, and R₅ is hydrogen.

In a sixteenth specific embodiment of the compound of formula (I), R₁ is hydrogen, R₂ is methoxy, R₃ is ethyl, R₄ is methoxy, and R₅ is hydrogen.

In a seventeenth specific embodiment of the compound of formula (I), R₁ is hydrogen, R₂ is methoxy, R₃ is n-propyl, R₄ is methoxy, and R₅ is hydrogen.

In an eighteenth specific embodiment of the compound of formula (I), R₁ is hydrogen, R₂ is methoxy, R₃ is fluoro, R₄ is methoxy, and R₅ is hydrogen.

In a nineteenth specific embodiment of the compound of formula (I), R₁ is hydrogen, R₂ is methoxy, R₃ is nitro, R₄ is methoxy, and R₅ is hydrogen.

In a twentieth specific embodiment of the compound of formula (I), R₁ is hydrogen, R₂ is methoxy, R₃ is ethylthio, R₄ is methoxy, and R₅ is hydrogen.

In a twenty-first specific embodiment of the compound of formula (I), R₁ is hydrogen, R₂ is methoxy, R₃ is isopropylthio, R₄ is methoxy, and R₅ is hydrogen.

In a twenty-second specific embodiment of the compound of formula (I), R₁ is hydrogen, R₂ is methoxy, R₃ is n-propylthio, R₄ is methoxy, and R₅ is hydrogen.

In a twenty-third specific embodiment of the compound of formula (I), R₁ is hydrogen, R₂ is methoxy, R₃ is cyclopropylmethylthio, R₄ is methoxy, and R₅ is hydrogen.

In a twenty-fourth specific embodiment of the compound of formula (I), R₁ is hydrogen, R₂ is methoxy, R₃ is tert-butylthio, R₄ is methoxy, and R₅ is hydrogen.

In a twenty fifth specific embodiment of the compound of formula (I), R₁ is hydrogen, R₂ is methoxy, R₃ is 2-fluoroethylthio, R₄ is methoxy, and R₅ is hydrogen.

In a twenty sixth specific embodiment of the compound of formula (I), R₂ and R₃ together with the carbon atoms that carry R₂ and R₃, respectively, form

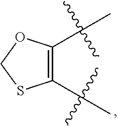 [SMILES: *C1=C(*)SCO1]

wherein the carbon atom carrying R₃ is connected to an S atom. R₁, R₄ and R₅ are as in formula (I).

The compound of formula (I) may be a compound of formula (II):

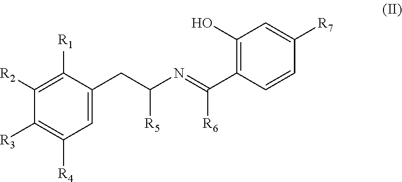

or a pharmaceutically acceptable salt thereof.

In formula (II), R₁, R₂, R₃, R₄, and R₅ are defined as in formula (I), including any one of specific embodiments of the compound of formula (I), as defined hereinabove. R₆ and R₇ are defined as in formula (I).

In a first specific embodiment of the compound of formula (II), R₆ is methyl and R₇ is hydrogen.

In a second specific embodiment of the compound of formula (II), R₆ is 4-chlorophenyl and R₇ is methoxy.

In a third specific embodiment of the compound of formula (II), R₆ is phenyl and R₇ is methoxy.

In a fourth specific embodiment of the compound of formula (II), R₆ is phenyl and Rr is hydrogen.

The compounds of formula (II) are particularly advantageous with respect to their ability to cross the blood-brain-barrier due to their enhanced nonpolar characteristics, while they still remain easily degradable by endogenous enzymes.

The compound of formula (I) may be a compound of formula (III):

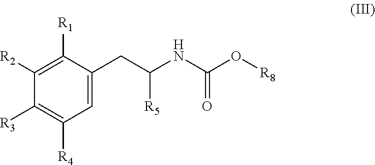

or a pharmaceutically acceptable salt thereof.

In formula (III), R₁, R₂, R₃, R₄, and R₅ are defined as in formula (I), including any one of specific embodiments of the compound of formula (I), as defined hereinabove. R₈ is defined as in formula (I).

In a first specific embodiment of the compound of formula (III), R₈ is ethyl.

In a second specific embodiment of the compound of formula (III), R₈ is isopropyl.

In a third specific embodiment of the compound of formula (III), R₈ is isobutyl.

In a fourth specific embodiment of the compound of formula (III), R₈ is tert-butyl.

In a fifth specific embodiment of the compound of formula (III), R₈ is neopentyl.

The compound of formula (I) may be a compound of formula (IV):

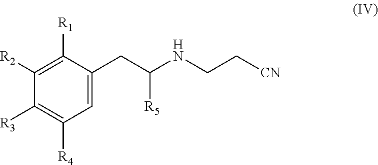

or a pharmaceutically acceptable salt thereof.

In formula (IV), R₁, R₂, R₃, R₄, and R₅ are defined as in formula (I), including any one of specific embodiments of the compound of formula (I), as defined hereinabove.

The compound of formula (I) may be a compound of formula (V):

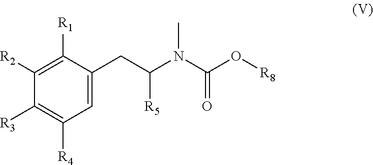

or a pharmaceutically acceptable salt thereof.

In formula (V), R₁, R₂, R₃, R₄, R₅ and R₈ are defined as in formula (I), including any one of specific embodiments of the compound of formula (I), as defined hereinabove.

In a first specific embodiment of the compound of formula (V), R₈ is ethyl.

In a second specific embodiment of the compound of formula (V), R₈ is isopropyl.

In a third specific embodiment of the compound of formula (V), R₈ is isobutyl.

In a fourth specific embodiment of the compound of formula (V), R₈ is tert-butyl. In this fourth specific embodiment of the compound of formula (V), preferably R₁ is hydrogen, R₂ and R₃ together with the carbon atoms that carry R₂ and R₃, respectively, form a five membered ring being

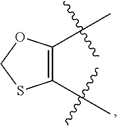 [SMILES: *C1=C(*)SCO1]

R₄ is hydrogen, and R₅ is methyl.

In a fifth specific embodiment of the compound of formula (V), R₈ is neopentyl.

In a sixth specific embodiment of the compound of formula (V), R₈ is methyl. In this sixth specific embodiment of the compound of formula (V), preferably R₁ is hydrogen, R₂ and R₃ together with the carbon atoms that carry R₂ and R₃, respectively, form a five membered ring being

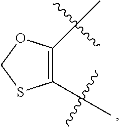 [SMILES: *C1=C(*)SCO1]

R₄ is hydrogen, and R₅ is methyl.

The compound of formula (I) may be a compound of formula (VI):

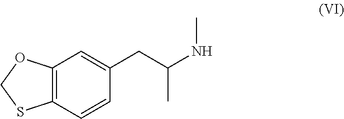

or a pharmaceutically acceptable salt thereof.

The compound of formula (I) may be a compound of formula (VII):

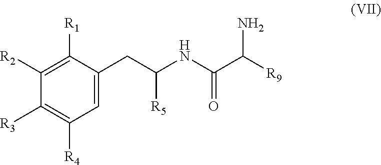

or a pharmaceutically acceptable salt thereof;
or it may be a compound of formula (VIII):

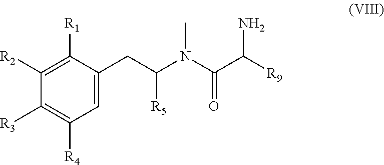

or a pharmaceutically acceptable salt thereof.

In formula (VII) or in formula (VIII), R₁, R₂, R₃, R₄, R₅ and R₉ are defined as in formula (I), including any one of the specific embodiments of the compound of formula (I), as defined hereinabove. Preferably, in formula (VII) or in formula (VIII), R₁ is hydrogen, R₂ and R₃ together with the carbon atoms that carry R₂ and R₃, respectively, form a five membered ring being

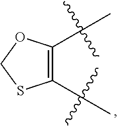 [SMILES: *C1=C(*)SCO1]

and R₄ is hydrogen. Preferably, the carbon atom carrying R₃ is connected to the S atom in said group

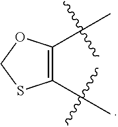 [SMILES: *C1=C(*)SCO1]

Preferably, in formula (VII) or in formula (VIII), R₅ is methyl.

In a first specific embodiment of the compound of formula (VII) or the compound of formula (VIII), R₉ is hydrogen.

In a second specific embodiment of the compound of formula (VII) or the compound of formula (VIII), R₉ is selected from methyl, ethyl, n-propyl, isopropyl, n-butyl, isobutyl, sec-butyl, tert-butyl and n-pentyl. Preferably, in this second specific embodiment, R₉ is selected from methyl, isopropyl, isobutyl, and sec-butyl.

In a third specific embodiment of the compound of formula (VII) or the compound of formula (VIII), R₉ is selected from —CH₂-phenyl, —CH₂-(4-hydroxyphenyl), —CH₂-(1H-imidazol-4-yl) and —CH₂-(1H-indol-3-yl).

In a fourth specific embodiment of the compound of formula (VII) or the compound of formula (VIII), R₉ is selected from —CH₂—OH, —CH(—CH₃)—OH, —CH₂—SH, and —CH₂CH₂—S—CH₃.

In a fifth specific embodiment of the compound of formula (VII) or the compound of formula (VIII), R₉ is selected from —CH₂—COOH, —CH₂CH₂—COOH, —CH₂—CO—NH₂ and —CH₂CH₂—CO—NH₂.

In a sixth specific embodiment of the compound of formula (VII) or the compound of formula (VIII), R₉ is selected from —(CH₂)₃—NH₂, (CH₂)₄—NH₂, —(CH₂)₄—NH—C(═NH)—NH₂ and —(CH₂)₃—NH—C(═NH)—NH₂.

It will be understood that in each one of formulae (II) to (VIII), the groups/variables (such as R₁, R₂, R₃ etc.) have the same meaning as the corresponding groups/variables (with the same name) comprised in formula (I), unless explicitly indicated otherwise.

A particularly preferred compound of formula (I) in accordance with the present invention is any one of the following compounds or a pharmaceutically acceptable salt thereof:

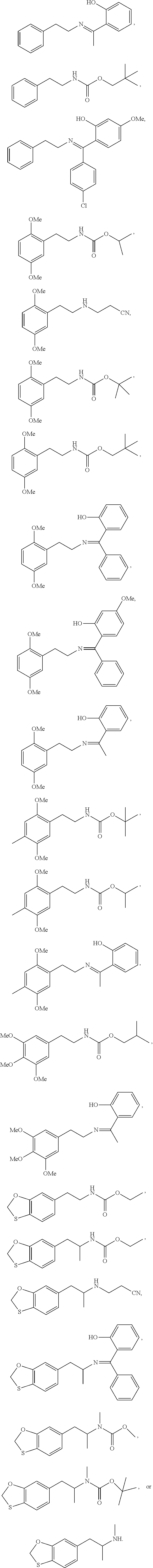

A further particularly preferred compound of formula (I) in accordance with the present invention is any one of the following compounds or a pharmaceutically acceptable salt thereof:

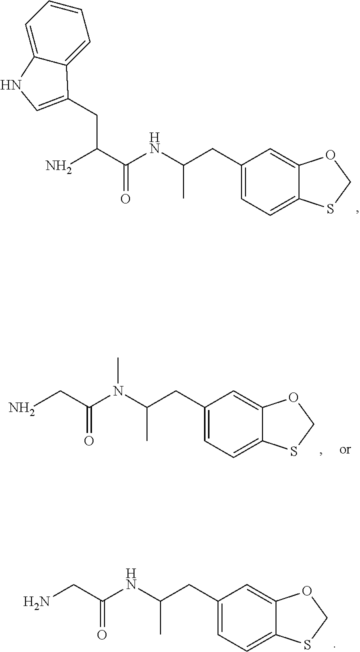 [SMILES: CC(Cc1ccc2c(c1)OCS2)NC(=O)C(N)Cc1c[nH]c2ccccc12 ; CC(Cc1ccc2c(c1)OCS2)N(C)C(=O)CN ; CC(Cc1ccc2c(c1)OCS2)NC(=O)CN]

Due to their specific molecular structure as a “prodrug”, i.e. as an active compound to be converted into its active form only within the body, the compounds of formula (I) presented herein have advantageous pharmacological properties. Accordingly, the phenethylamine prodrugs according to the present invention are pharmacologically released, taken up and metabolized in the human body with different pharmacokinetics (as compared to the original phenethylamines). The potential for addiction and the neurotoxic effect of psychotropic substances are often related to a rapid increase of their concentration upon uptake of the substances. Therefore, active compounds leading only to a slow increase from the initial concentration are beneficial from a pharmaceutical point of view.

The compounds of formula (I) according to the invention exert their effect on the organism only after endogenous metabolization into the actual active phenethylamine compounds (such as PEA, 2C-D or mescaline), whereby delayed pharmacokinetics and a longer-lasting effect are obtained. The steadier and more uniform release of the active compound in the organism furthermore contributes to reducing side effects. The “depot effect” resulting from such delayed release is therefore a particular advantage of the present invention. It is further noted that the compounds of the present invention are particularly advantageous with respect to their ability to cross the blood-brain-barrier due to their enhanced nonpolar characteristics while still remaining easily degradable by endogenous enzymes. These chemical alterations could also lead to retarded enzyme kinetics being especially valuable for fast metabolized compounds like phenethylamine (PEA) which are prone to be inactivated within minutes. Furthermore the effective dose of drugs like mescaline known for its noticeable side effects (related to the high minimum effective dose) could be reduced by these altered transportation and metabolism kinetics due to the new prodrug characteristics.

The compounds of formula (I) can be prepared in accordance with, or in analogy to, the synthetic routes described in the examples section. General synthesis routes and considerations regarding the synthesis of the compounds of formula (II), (II) and (IV) are discussed in the following. Specific examples of preparation of the compounds of formula (V) and (VI) are shown in the Examples.

The compounds of formula (II) can be prepared according to the general method comprising the following steps:
a. Preparing a solution of phenethylamine in solvent 1
b. Adding a ketone compound and a catalyst (e.g. p-TsOH) under protective gas atmosphere
c. Stirring of the mixture for at least 12 hours at 150° C. with dean-stark-apparatus
d. Purificating of reaction mixture by filtration through silica plug by using hexane/ethyl acetate 8:2
e. Obtaining the phenethylimine according to the invention

In step a and b., between 1 mmol and 3 mmol of a ketone compound (which may be selected, e. g. from the group consisting of 2′-Hydroxyacetophenone, 2-Hydroxybenzophenone, 2-Hydroxy-4-methoxybenzophenone and 4′-Chloro-2-hydroxy-4-methoxybenzophenone) and the corresponding phenethylamine (between 1 mmol and 3 mmol), is dissolved in 10 ml up to 30 ml of solvent I, wherein solvent I is, e. g. toluene, benzene or xylene.

The catalyst in step b is preferably selected from a group consisting of para-toluenesulfonic acid, methanesulfonic acid, sulfuric acid, boron trifluoride etherate and titanium tetrachloride. Particularly preferred catalyst in step b is para-toluenesulfonic acid (p-TsOH).

The solution obtained is aerated with protective gas. The term “protective gas”, as used herein, refers to an inert gas, preferably argon. It is however noted and apparent to the skilled person that also a different protective gas can be employed, e. g. elementary gases such as nitrogen, noble gases such as helium, neon, argon, krypton, xenon, and gaseous molecular compounds like sulfur hexafluoride.

This solution is stirred, e. g. at a temperature between 90° C. and 160° C., preferably at 150° C. (423.15 Kelvin).

In step d., the crude product is further purified. The purification can be conducted, e. g. by column purification over silica using the eluent mixture hexane/ethyl acetate, e. g. in a ratio of 8:2 in one embodiment. Other column materials and eluents known in the art can also be used.

With this method, in step e., the product can be obtained in yields of more than 70 wt-% (gravimetric determination of the amount of the end product, relative to the intermediate product).

Further details on the method of production are provided in the examples and will be apparent to the person skilled in the art.

The compounds of formula (III) can be obtained according to the method comprising the following steps:
a. Preparing a solution of phenethylamine in solvent I
b. Adding of activating agent under protective gas atmosphere
c. Adding of derivatization agent under protective gas atmosphere
d. Stirring of the mixture under protective gas atmosphere for at least 2 hours at room temperature
e. Stopping the reaction by dilution with solvent (e. g. solvent I from step (a))
f. Extracting with water and saturated saline solution
g. Drying the organic phase over a desiccant at 40-60° C. under vacuum (or reduced pressure)
h. Obtaining the phenethylcarbamate according to the invention

In step (a), between 1 mmol and 8 mmol of phenethylamine are dissolved in 5 ml to 30 ml of solvent I, wherein solvent I is selected from tetrahydrofuran, dioxane, 2-methyltetrahydrofuran, chloroform and dichloromethane.

This can be done at a temperature between −78° C. and 45° C., preferably at a temperature between 5° C. and 40° C., more preferably at room temperature (293.15 Kelvin; 20° C.).

In step (b), between 1.2 mmol and 3.6 mmol of an activating agent are added, such as, e.g., a nitrogen base. In this case, the nitrogen base is selected from triethylamine, diisopropyl ethylamine, pyridine, diazabicyloundecene, diazabicyclononene and 4-dimethyl aminopyridine. It is also possible to use a deprotonating agent such as n-butyllithium (n-BuLi).

In step (c), between 1.1 mmol and 3.3 mmol of a derivatization agent are added dropwise through a septum, wherein the derivatization agent is selected from ethyl chloroformate, di-tert-butyl pyrocarbonate, isopropylchloroformate, isobutylchloroformate and neopentylchlorofomate.

In step (d), the mixture is stirred between 2 and 10 hours at 20-28° C. under protective gas atmosphere.

In step (e), the reaction is stopped by adding between 10 ml and 70 ml of the solvent I from step (a).

In step (f), extraction is performed with between 10 ml and 100 ml of water. Subsequent extraction with between 10 ml and 100 ml saturated saline solution may be performed.

In step (g), the mixture is dried. Particularly preferred is drying with a desiccant at a temperature between 35° C. and 60° C. and a vacuum (reduced pressure) of 30-60 mbar. Preferred desiccants are anhydrous calcium chloride, anhydrous sodium carbonate, anhydrous potassium carbonate, anhydrous sodium sulfate, anhydrous magnesium sulphate, or anhydrous calcium sulfate. Particularly preferred desiccant is anhydrous MgSO4, the temperature is 45° C. and the vacuum is 40 mbar.

The product obtained in steps (a) to (h) contains the phenethylcarbamate derivative of formula (II) according to the invention.

With this method, in step h., the product can be obtained in yields of more than 90 wt-% (gravimetric determination of the amount of the end product, relative to the intermediate product).

Further details on the method of production are provided in the examples and will be apparent to the person skilled in the art.

The compound of formula (IV) can be obtained according to the method comprising the following steps:
a. Preparing a solution of phenethylamine in solvent I
b. Adding of activating agent under protective gas atmosphere
c. Adding of derivatization agent under protective gas atmosphere
d. Stirring of the mixture under protective gas atmosphere for at least 12 hours at room temperature
e. Stopping the reaction by dilution with solvent (e.g., solvent I from step (a))
f. Extracting with water and saturated saline solution
g. Drying the organic phase over a desiccant at 40-60° C. under vacuum (or reduced pressure)
h. Obtaining the crude product
i. Purifying the crude product by column chromatography
j. Obtaining the phenethylamine prodrug of formula (IV) according to the invention

In step (a), between 1 mmol and 2 mmol of phenethylamine are dissolved in 5 ml to 10 ml of solvent I, wherein solvent I is selected from tetrahydrofuran, dioxane, 2-methyltetrahydrofuran, chloroform and dichloromethane.

This can be done at a temperature between −78° C. and 45° C., preferably at a temperature between 5° C. and 40° C., more preferably at room temperature (293.15 Kelvin; 20° C.).

In step (b), between 1.3 mmol and 2.6 mmol of an activating agent are added, such as e. g. a nitrogen base. The nitrogen base is preferably selected from triethylamine, diisopropyl ethylamine, pyridine, diazabicyloundecene, diazabicyclononene and 4-dimethyl aminopyridine. It is also possible to use a deprotonating agent such as n-butyllithium (n-BuLi).

In step (c), between 1.1 mmol and 2.2 mmol of a derivatization agent are added dropwise through a septum, wherein the derivatization agent is selected from 3-bromopropionitrile, 1-bromo-3-chloropropane and 3-bromoprop-1-yne.

In step (d), the mixture is stirred between 24 and 36 hours at 20-28° C. under protective gas atmosphere.

In step (e), the reaction is stopped by adding between 10 ml and 20 ml of the solvent I from step (a).

In step (f), extraction is performed with between 10 ml and 50 ml of water. The subsequent extraction with between 10 ml and 50 ml saturated saline solution may performed.

In step (g), the mixture is dried. Particularly preferred is drying with a desiccant at a temperature between 35° C. and 60° C. and a vacuum (reduced pressure) of 30-60 mbar. Preferred desiccants are anhydrous calcium chloride, anhydrous sodium carbonate, anhydrous potassium carbonate, anhydrous sodium sulfate, anhydrous magnesium sulphate, or anhydrous calcium sulfate. Particularly preferred desiccant is anhydrous MgSO4, the temperature is 45° C. and the vacuum is 40 mbar.

In step i., the crude product is further purified. The purification can be conducted, e. g. by column purification over silica using the eluent acetone in one embodiment. Other column materials and eluents known in the art can also be used.

The product obtained in steps (a) to (j) contains the compound of formula (IV) according to the invention.

With this method, in step h., the product can be obtained in yields of more than 55 wt-% (gravimetric determination of the amount of the end product, relative to the intermediate product).

Methods of production of the compounds of formula (VII) or (VIII) are also provided herein, and may comprise the following steps:
a. preparing a solution of a protected amino acid in solvent I (THF or dichloromethane);
b. addition of an activating agent dissolved in solvent I under protective gas atmosphere;
c. stirring of the mixture under protective gas atmosphere for at least 2 hours at room temperature;
d. phenethylamine (as a free base) dissolved in solvent I is added dropwise under protective gas atmosphere;
e. stirring of the mixture under protective gas atmosphere for at least 2 hours at room temperature;
f. stopping the reaction by adding 2% ammonia solution;
g1. concentration of the solvent I;
g2. dissolving the residue in ethyl acetate;
h. extraction with 1M HCl, water and saturated saline solution;
i. drying of the organic phase over a desiccant at 40-60° C. and under vacuum;
j. obtaining the crude product;
k. purification of the crude product by recrystallization and/or column chromatography;
l. obtaining the protected phenethylamine peptide;
m. deprotection of the protected phenethylamine peptide;
n. purification of the phenethylamine peptide according to the invention by means of column chromatography;
o. obtaining the phenethylamine peptide according to the invention.

In step a., between 4.5 mmol and 14.5 mmol of a protected amino acid, which may be selected, e.g., from the group consisting of N-(9-fluorenylmethyloxycarbonyl)-L-tryptophan and N-(9-fluorenylmethyloxycarbonyl)-L-glycine, is dissolved in 33 ml up to 100 ml of solvent I, wherein solvent I is, e.g., selected from the group consisting of tetrahydrofuran, dioxane, 2-methyltetrahydrofuran, or dichloromethane.

This can be done, e.g., at a temperature between 5° C. and 40° C., preferably at room temperature (293.15 Kelvin; 20° C.).

The solution obtained is aerated with protective gas.

The term “protective gas”, as used herein, refers to an inert gas, preferably argon. In other embodiments, also a different protective gas can be employed, e.g., elementary gases such as nitrogen, noble gases such as helium, neon, argon, krypton, xenon, and gaseous molecular compounds like sulfur hexafluoride.

In step b., between 5 mmol and 16 mmol of an activating agent, such as, e.g., 1,1′-carbonyldiimidazole or a combination of a nitrogen base and a carbodiimide, dissolved in 13 ml to 40 ml of solvent I, are added dropwise. In this case, in preferred embodiments, the nitrogen base is selected from the group consisting of triethylamine, diisopropyl ethylamine, pyridine, and 4-dimethyl aminopyridine. The carbodiimide, which may be added, is preferably selected from the group consisting of dicyclohexyl carbodiimide (DCC), diisopropyl carbodiimide (DIC), 1-ethyl-3-(3-dimethylaminopropyl) carbodiimide (EDC), 1-[bis(dimethylamino)methylene]-1H-1,2,3-triazolo[4,5-b]pyridinium 3-oxide hexafluorophosphate (HATU), or (benzotriazol-1-yloxy)tripyrrolidinophosphonium hexafluorophosphate (PyBOP).

In step c., the mixture may be stirred between 2 and 3 hours at 20-28° C. under protective gas atmosphere.

In step d., between 5 mmol and 16 mmol of a phenethylamine (free base), for example selected from the group consisting of Benzoxathiol-6-yl-propan-2-amine, Benzoxathiol-6-yl-N-methyl-propan-2-amine and Benzoxathiol-6-yl-ethan-1-amine, dissolved in 8 ml up to 15 ml of solvent I, may be added dropwise through a septum.

In step e., the mixture may be stirred between 1 and 3 hours at 20-28° C. under protective gas atmosphere. In one embodiment, it is stirred for at least 0.5 hours and up to 4 hours; and/or at 20° C. under protective gas atmosphere.

In step f., the reaction may be stopped by adding between 10 ml and 15 ml of 2% ammonia solution.

In step g., the mixture may be dried, preferably in a rotatory evaporator under vacuum (i.e., at reduced pressure) (step g1.), and is redissolved in solvent II (e.g., in 400 ml to 600 ml of solvent II) (step g2.), wherein solvent II is preferably selected from the group consisting of diethyl ether, methyl-tert-butyl ether, ethyl acetate, chloroform, and dichloromethane.

In step h., extraction may be performed with between 100 ml and 150 ml of 0.5 molar hydrochloric acid. In one embodiment, subsequent extraction with between 100 ml and 150 ml water is performed. In one embodiment, subsequent extraction with between 100 ml and 150 ml saturated saline solution is performed.

In step i., the mixture may be dried. Particularly preferred is drying with a desiccant at a temperature between 35° C. and 60° C. and a vacuum (reduced pressure) of 30-60 mbar.

Preferred desiccants are anhydrous calcium chloride, anhydrous sodium carbonate, anhydrous potassium carbonate, anhydrous sodium sulfate, anhydrous magnesium sulfate, or anhydrous calcium sulfate. In one embodiment, the desiccant is anhydrous MgSO₄, the temperature is 45° C., and the vacuum (reduced pressure) is 40 mbar.

The crude product obtained in steps a. to j. contains the intermediate product of the protected amide prodrug according to the invention.

In step k., the crude product is further purified. The purification can be conducted, e.g., by dissolving the crude product in toluene/ethanol at 10:1 with subsequent evaporation at 50° C. and 400 mbar until crystallization and/or column purification over silica using the eluent mixture hexane/ethyl acetate, e.g. in a ratio of 1:1 in one embodiment. Other column materials and eluents known in the art can also be used.

With this method, the intermediate product of step l. can be obtained in yields of more than 60 wt-% (gravimetric determination of the amount of the end product, relative to the starting materials).

In step m., between 1.6 mmol and 10 mmol of the protected amide prodrug may be dissolved in 75 ml to 300 ml of solvent I and 3.2 mmol to 20 mmol piperidine are added dropwise. Stirring is conducted for between 2 and 24 hours at 25° C. under protective gas atmosphere. Alternatively, when using carbobenzoxy-protected amino acids, cleaving the protective group may be carried out via a catalytic hydration using palladium on activated carbon in ethanol as a solvent.

In step n., the deprotected amide prodrug may be further purified. The purification can be conducted by column chromatographic purification over silica using the eluent mixture dichloromethane/methanol with 1% ammonia, e.g., in a ratio of 9:1 in one embodiment. Other column materials and eluents known in the art can also be used.

With this method, in step o., the product can be obtained in yields of more than 80 wt-% (gravimetric determination of the amount of the end product, relative to the intermediate product).

The synthetic methodology for the compounds of formula (VII) and (VIII) is also applicable for the compounds wherein
X is

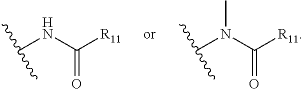

In n the case of the amide prodrugs, i.e. the compounds of formula (VII) and (VIII), the resulting betaine structure thus provides for better uptake of the phenethylamine peptides.

Further details on the method of production are provided in the examples and will be apparent to the person skilled in the art.

The following definitions apply throughout the present specification and the claims, unless specifically indicated otherwise.

The term “hydrocarbon group” refers to a group consisting of carbon atoms and hydrogen atoms.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₆ alkyl” denotes an alkyl group having 1 to 6 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₆ alkenyl” denotes an alkenyl group having 2 to 6 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₆ alkynyl” denotes an alkynyl group having 2 to 6 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₆ alkylene” denotes an alkylene group having 1 to 6 carbon atoms, and the term “C₀₋₆ alkylene” indicates that a covalent bond (corresponding to the option “C₀ alkylene”) or a C₁₋₆ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkenyl or cycloalkyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkenyl or heterocycloalkyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from 0, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members (e.g., cyclopropyl or cyclohexyl).

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, 1,1-dioxothianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I).

As used herein, the term “nitro” refers to a group —NO₂.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a fumarate salt, a maleate salt, an oxalate salt, a malate salt, a tartrate salt, and a mesylate salt.

The present invention also specifically relates to the compound of formula (I), including any one of the specific compounds of formula (I) described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates and non-racemic mixtures). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I). It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The present invention relates to a pharmaceutical composition comprising a compound of formula (I) or a pharmaceutically acceptable salt thereof of the present invention as defined herein, and a pharmaceutically acceptable excipient. The present invention further relates to the said pharmaceutical composition comprising a compound of formula (I) or a pharmaceutically acceptable salt thereof of the present invention, and a pharmaceutically acceptable excipient for use in therapy.

The compounds and/or chemical entities provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the pharmaceutically acceptable salts thereof or the above described pharmaceutical compositions comprising the compound of formula (I) or the pharmaceutically acceptable salt thereof may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutically acceptable salts or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutically acceptable salts or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds, the pharmaceutically acceptable salts or the pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds, pharmaceutically acceptable salts or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds, pharmaceutically acceptable salts or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention or a pharmaceutically acceptable salt thereof.

Said compounds, pharmaceutically acceptable salts or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) or the pharmaceutically acceptable salt thereof for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds, pharmaceutically acceptable salts or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds, pharmaceutically acceptable salts or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Preferred routes of administration are oral administration or parenteral administration. For each of the compounds, salts or pharmaceutical compositions provided herein, it is particularly preferred that the respective compound or pharmaceutical composition is to be administered orally (particularly by oral ingestion).

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The invention further relates to the compound of formula (I) or the pharmaceutically acceptable salt thereof or to the for use as a medicament. As understood herein the use as a medicament is meant as the use in the treatment of a disease.

As used herein, the term “treatment” (or “treating”) in relation to a disease or disorder refers to the management and care of a patient for the purpose of combating the disease or disorder, such as to reverse, alleviate, inhibit or delay the disease or disorder, or one or more symptoms of such disease or disorder. It also refers to the administration of a compound or a composition for the purpose of preventing the onset of symptoms of the disease or disorder, alleviating such symptoms, or eliminating the disease or disorder. Preferably, the “treatment” is curative, ameliorating or palliative.

The compounds of formula (I) or the pharmaceutically acceptable salts thereof, or the pharmaceutical compositions of the present invention are useful in the treatment of a serotonin 5-HT_{2A} receptor associated disease/disorder.

A serotonin 5-HT_{2A} receptor associated disease/disorder as described herein, is preferably an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, Alzheimer's disease or dementia. More preferably, a serotonin 5-HT_{2A} receptor associated disease/disorder is an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, or vomiting

The subject or patient to be treated in accordance with the present invention may be an animal (e.g., a non-human animal). Preferably, the subject/patient is a mammal. More preferably, the subject/patient is a human (e.g., a male human or a female human) or a non-human mammal. Most preferably, the subject/patient to be treated in accordance with the invention is a human.

The invention will now be described by reference to the following examples which are merely illustrative and are not to be construed as a limitation of the scope of the present invention.

### EXAMPLES

### Preparative Example 1: Neopentyl-phenethylcarbamate

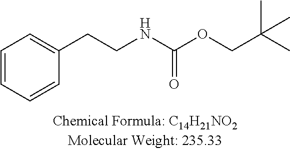 [SMILES: CC(C)(C)COC(=O)NCCc1ccccc1]

Phenethylamine (3 mmol/363 mg) was dissolved in dichloromethane (7 ml) at 25° C. Triethylamine (3.6 mmol/0.49 ml) was added and aerated with argon. This results in a clear solution. Neopentyl chloroformate (3.3 mmol/0.49 ml) was added dropwise through septum. Upon addition, a whitish haze in the solution forms immediately. Stirring for 1.5 h under argon at 25° C.

Inprocess-control via TLC shows quantitative conversion to the requested product.

The reaction solution was diluted with dichloromethane (30 ml) and filtered through a plug of silica. After evaporation of the combined product-fractions, a colorless oil was obtained (700 mg, 99%).

LC-MS measurement for the obtained compound are shown in FIG. 1.

The LC-MS measurements for this as well as other examples described herein have been performed as in the following.

LC-system:
Running time: 9.0 min
Column: Shimadzu Shim-pack Scepter C18 1.9 micrometer;
dimension 100 × 2.1 mm
Oven temperature: 40° C.
UV-detection: 215-400 nm
Gradient: solvent A = water; solvent B = acetonitrile

| | Flow | A | B | Time |
|---|---|---|---|---|
| 1 | 0.6 ml/min | 95% | 5% | |
| 2 | 0.6 ml/min | 95% | 5% | 1.50 min |
| 3 | 0.6 ml/min | 15% | 85% | 7.00 min |
| 4 | 0.6 ml/min | 3% | 97% | 7.02 min |
| 5 | 0.6 ml/min | 3% | 97% | 7.50 min |
| 6 | 0.6 ml/min | 95% | 5% | 7.52 min |

MS-system:
Running time: 9.0 min
Acqusition mode: Scan positive
Start m/z: 90 End m/z: 900
Scan speed: 10000 u/sec
Event time: 0.1 sec

### Preparative Example 2: N-Phenethyl-2-hydroxy-acetophenonimine

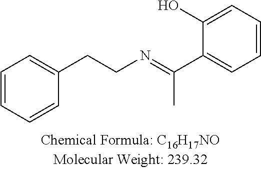 [SMILES: C/C(=N\CCc1ccccc1)c1ccccc1O]

Phenethylamine (2 mmol/242 mg) was diluted in toluene (12 ml) and 2-hydroxy-acetophenone (2 mmol/0.24 ml) was added. After installation of a dean-stark-apparatus, a catalytic amount of p-toluenesulfonic acid (0.06 mmol/12 mg) was added and the reaction mixture was stirred at 140° C.

After stirring for 30 h under argon, inprocess-control via TLC shows quantitative conversion to the requested product. The reaction mixture was evaporated to a final volume of approx. 2 ml.

The crude product in toluene was filtered through a plug of silica. After evaporation of the combined product-fractions, a yellow oil was obtained (370 mg, 77%).

LC-MS measurements of the obtained compound are shown in FIG. 2.

### Preparative Example 3: N-Phenethyl-2-hydroxy-4-methoxy-4′-chloro-benzophenonimine

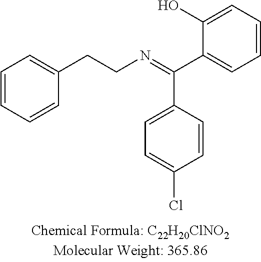 [SMILES: Oc1ccccc1/C(=N/CCc1ccccc1)c1ccc(Cl)cc1]

Phenethylamine (1 mmol/121 mg) was diluted in toluene (6 ml) and 4′-Chloro-2-hydroxy-4-methoxybenzophenone (1 mmol/262 mg) was added. After installation of a dean-stark-apparatus, a catalytic amount of p-toluenesulfonic acid (0.03 mmol/6 mg) was added and the reaction mixture was stirred at 130° C.

After stirring for 3 d under argon, inprocess-control via TLC shows quantitative conversion to the requested product. The reaction mixture was evaporated to a final volume of approx. 2 ml.

The crude product in toluene was filtered through a plug of silica. After evaporation of the combined product-fractions, a brown solid was obtained (330 mg, 90%).

LC-MS measurements of the obtained compound are shown in FIG. 3.

### Preparative Example 4: Isobutyl (3,4,5-trimethoxyphenethyl)carbamate

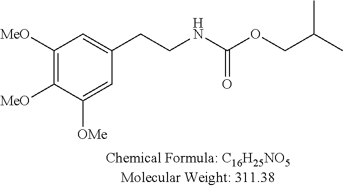 [SMILES: COc1cc(CCNC(=O)OCC(C)C)cc(OC)c1OC]

3,4,5-Trimethoxyphenethylamine (2 mmol/422 mg) was dissolved in dichloromethane (7 ml) at 25° C. Triethylamine (2.4 mmol/0.33 ml) was added and aerated with argon. This results in a clear solution. Isobutyl chloroformate (2.2 mmol/0.28 ml) was added dropwise through septum. Upon addition, a whitish haze in the solution forms immediately. Stirring for 3 h under argon at 25° C.

Inprocess-control via TLC shows quantitative conversion to the requested product.

The reaction solution was diluted with dichloromethane (30 ml) and filtered through a plug of silica. After evaporation of the combined product-fractions, a colorless oil was obtained (510 mg, 82%).

LC-MS measurements of the obtained compound are shown in FIG. 4.

### Preparative Example 5: Isopropyl (2,5-Dimethoxyphenethyl)carbamate

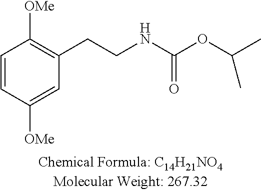 [SMILES: COc1ccc(OC)c(CCNC(=O)OC(C)C)c1]

2,5-Dimethoxyphenethylamine (3 mmol/543 mg) was dissolved in dichloromethane (10 ml) at 4° C. Triethylamine (3.6 mmol/0.5 ml) was added and aerated with argon. This results in a clear solution. Isopropyl chloroformate (3.3 mmol/3.3 ml 1M in toluene) was added dropwise through septum.

After stirring for 30 min under argon at 4° C., inprocess-control via TLC shows quantitative conversion to the requested product.

The reaction solution was diluted with dichloromethane (40 ml) and filtered through a plug of silica. After evaporation of the combined product-fractions, a colorless oil was obtained (770 mg, 96%).

LC-MS measurements of the obtained compound are shown in FIG. 5.

### Preparative Example 6: (2,5-Dimethoxyphenethyl-amino)propanenitrile

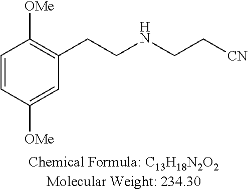 [SMILES: COc1ccc(OC)c(CCNCCC#N)c1]

2,5-Dimethoxyphenethylamine (2 mmol/362 mg) was dissolved in dichloromethane (5 ml) at 25° C. Diazabicycloundecene (2.6 mmol/0.39 ml) was added and aerated with argon. 3-Bromo-propanenitrile (2.2 mmol/0.18 ml) was added dropwise through septum. After stirring for 18 h under argon at 25° C., inprocess-control via HPLC shows 90% conversion to the requested product.

After stirring for 24 h at 25° C. the reaction solution was diluted with dichloromethane (20 ml) and extracted with 10 ml water and 10 ml saturated saline solution. Subsequently, the organic phase was dried over some MgSO4. Subsequently, the organic phase was slowly concentrated on the rotary evaporator, yielding the raw product as a yellowish oil (450 mg).

The raw product was diluted in acetone and filtered through a plug of silica. After evaporation of the combined product-fractions, a yellowish oil was obtained (300 mg, 64%).

LC-MS measurements of the obtained compound are shown in FIG. 6.

### Preparative Example 7: tert.-butyl-(2,5-Dimethoxyphenethyl)carbamate

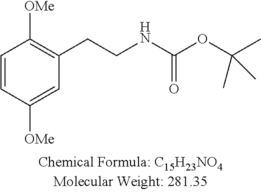 [SMILES: COc1ccc(OC)c(CCNC(=O)OC(C)(C)C)c1]

2,5-Dimethoxyphenethylamine (3 mmol/543 mg) was dissolved in dichloromethane (10 ml) at 4° C. Triethylamine (3.6 mmol/0.5 ml) was added and aerated with argon. Di-tert-butyl dicarbonate (3.3 mmol/719 mg diluted in tetrahydrofuran) was added dropwise through septum. This results in a clear colorless solution.

After stirring for 9 h under argon at 25° C., inprocess-control via HPLC shows quantitative conversion to the requested product. The reaction mixture was then diluted with dichloromethane (50 ml) and extracted with 50 ml water and 50 ml saturated saline solution. Subsequently, the organic phase was dried over some MgSO4. Subsequently, the organic phase was slowly concentrated on the rotary evaporator, yielding the raw product as a colorless solid (1.20 g).

This crude material was dissolved in ethanol (10 ml) at 45° C.

For purification, the material was recrystallized from ethanol at 45° C. To strengthen the formation of crystals, some ether was added after cooling. Following filtration, 430 mg of colorless solid (51%) was obtained.

LC-MS measurements for the obtained compound are shown in FIG. 7.

### Preparative Example 8: Ethyl-((benzoxathiol-6-yl)propan-2-yl)carbamate

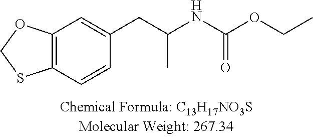 [SMILES: CCOC(=O)NC(C)Cc1ccc2c(c1)OCS2]

Benzoxathiol-6-yl-propan-2-amine (8.1 mmol/1.58 g) was dissolved in dichloromethane (30 ml) at 25° C. Triethylamine (10.4 mmol/1.45 ml) was added and aerated with argon. Ethylchloroformate (8.9 mmol/0.85 ml) was added dropwise through septum.

After stirring for 15 h under argon at 25° C., inprocess-control via TLC shows quantitative conversion to the requested product. The reaction mixture was then diluted with dichloromethane (70 ml) and extracted with 60 ml 0.1 N HCl and 60 ml saturated saline solution.

The raw product in dichloromethane was filtered through a plug of silica. After evaporation of the combined product-fractions, a yellow oil was obtained (1.20 g, 98%).

LC-MS measurements for the obtained compound are shown in FIG. 8.

### Preparative Example 9: Ethyl-(benzoxathiol-6-yl)ethyl)carbamate

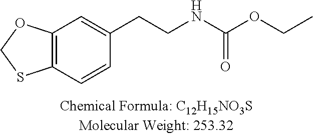 [SMILES: CCOC(=O)NCCc1ccc2c(c1)OCS2]

Benzoxathiol-6-yl-ethan-1-amine (0.6 mmol/0.1 g) was dissolved in dichloromethane (30 ml) at 25° C. Triethylamine (1.2 mmol/0.2 ml) was added and aerated with argon. Ethylchloroformate (1 mmol/0.1 ml) was added dropwise through septum.

After stirring for 16 h under argon at 25° C., inprocess-control via TLC shows quantitative conversion to the requested product.

The crude product in dichloromethane was filtered through a plug of silica. After evaporation of the combined product-fractions, a yellow oil was obtained (70 mg, 46%).

LC-MS measurements for the obtained compound are shown in FIG. 9.

### Preparative Example 10: Neopentyl-(2,5-Dimethoxyphenethyl)carbamate

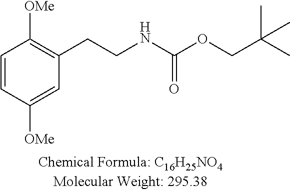 [SMILES: COc1ccc(OC)c(CCNC(=O)OCC(C)(C)C)c1]

2,5-Dimethoxyphenethylamine (1.4 mmol/250 mg) was dissolved in dichloromethane (30 ml) at 25° C. Triethylamine (1.8 mmol/0.25 ml) was added and aerated with argon. Neopentyl chloroformate (1.54 mmol/0.23 ml) was added dropwise through septum.

After stirring for 4 h under argon at 25° C., inprocess-control via TLC shows quantitative conversion to the requested product. The reaction mixture was then diluted with dichloromethane (30 ml) and extracted with 50 ml water and 50 ml saturated saline solution. Subsequently, the organic phase was dried over some MgSO4. Subsequently, the organic phase was slowly concentrated on the rotary evaporator, yielding the crude product as a colorless oil (1.60 g).

The crude product was treated on a column over 50 g silica using the eluent mixture hexane/methyl-tert. butyl ether in a ratio of 8:2. This yielded 380 mg the product as a colorless oil (91%).

LC-MS measurements for the obtained compound are shown in FIG. 10.

### Preparative Example 11: Benzoxathiol-6-yl-N-methyl-propan-2-amine

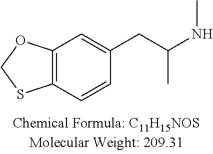

Ethyl-(benzoxathiol-6-yl)propan-2-yl)carbamate (6.53 mmol/1.74 g) was dissolved in tetrahydrofuran (30 ml) at 25° C. This carbamate-solution was then added dropwise to a refluxing solution of lithium aluminiumhydride (50 mmol) in 320 ml tetrahydrofuran. The reaction mixture was stirred at 80° C. for 5 h.

After stirring over night at 25° C., the reaction was stopped by the addition of sodium sulfate decahydrate (10 g). The resulting slurry was filtered and rinsed with tetrahydrofuran. After evaporation of the filtrate, a colorless oil was obtained (1.2 g).

The crude oil was treated on a column over 85 g silica using the eluent mixture dichloromethane/methanol in a ratio of 9:1. This yielded 580 mg the product as a colorless oil (42%).

LC-MS measurements for the obtained compound are shown in FIG. 11.

### Preparative Example 12: N-(2,5-Dimethoxyphenethyl)-2-hydroxy-benzophenonimine

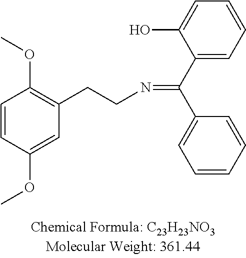 [SMILES: COc1ccc(OC)c(CC/N=C(\c2ccccc2)c2ccccc2O)c1]

2,5-Dimethoxyphenethylamine (3 mmol/543 mg) was diluted in toluene (30 ml) and 2-hydroxy-benzophenone (3 mmol/594 mg) was added. After installation of a dean-stark-apparatus, a catalytic amount of p-toluenesulfonic acid (0.1 mmol/36 mg) was added and the reaction mixture was stirred at 140° C.

After stirring for 20 h under argon, inprocess-control via TLC shows quantitative conversion to the requested product. The reaction mixture was evaporated to a final volume of approx. 10 ml.

The crude product in toluene was filtered through a plug of silica. After evaporation of the combined product-fractions, a yellow oil was obtained (940 mg, 85%).

LC-MS measurements for the obtained compound are shown in FIG. 12.

### Preparative Example 13: N-(2,5-Dimethoxyphenethyl)-2-hydroxy-4-methoxy-benzophenonimine

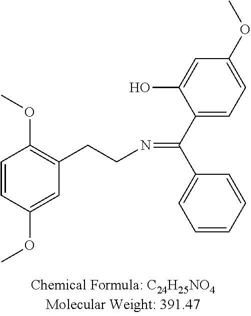 [SMILES: COc1ccc(/C(=N/CCc2cc(OC)ccc2OC)c2ccccc2)c(O)c1]

2,5-Dimethoxyphenethylamine (3 mmol/543 mg) was diluted in toluene (30 ml) and 2-hydroxy-4-methoxy-benzophenone (3 mmol/685 mg) was added. After installation of a dean-stark-apparatus, a catalytic amount of p-toluenesulfonic acid (0.1 mmol/36 mg) was added and the reaction mixture was stirred at 140° C.

After stirring for 20 h under argon, inprocess-control via TLC shows quantitative conversion to the requested product. The reaction mixture was evaporated to a final volume of approx. 10 ml.

The crude product in toluene was filtered through a plug of silica. After evaporation of the combined product-fractions, a yellow oil was obtained (790 mg, 66%).

### Preparative Example 14: N-(2,5-Dimethoxyphenethyl)-2-hydroxy-acetophenonimine

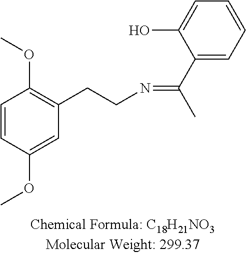 [SMILES: COc1ccc(OC)c(CC/N=C(\C)c2ccccc2O)c1]

2,5-Dimethoxyphenethylamine (3 mmol/543 mg) was diluted in toluene (30 ml) and 2-hydroxy-acetophenone (3 mmol/0.36 ml) was added. After installation of a dean-stark-apparatus, a catalytic amount of p-toluenesulfonic acid (0.1 mmol/36 mg) was added and the reaction mixture was stirred at 140° C.

After stirring for 22 h under argon, inprocess-control via TLC shows quantitative conversion to the requested product. The reaction mixture was evaporated to a final volume of approx. 10 ml.

The crude product in toluene was filtered through a plug of silica. After evaporation of the combined product-fractions, a yellow oil was obtained (790 mg, 88%).

LC-MS measurements for the obtained compound are shown in FIG. 13.

### Preparative Example 15: N-(benzoxathiol-6-yl)propan-2-yl)-2-hydroxy-benzophenonimine

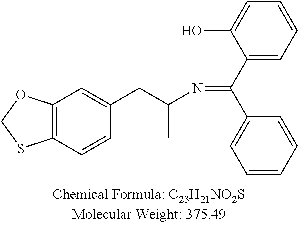 [SMILES: CC(Cc1ccc2c(c1)OCS2)/N=C(\c1ccccc1)c1ccccc1O]

Benzoxathiol-6-yl-propan-2-amine (1 mmol/197 mg) was dissolved in toluene (20 ml) and 2-hydroxy-benzophenone (1 mmol/195 mg) was added. After installation of a dean-stark-apparatus, a catalytic amount of p-toluenesulfonic acid (0.03 mmol/6 mg) was added and the reaction mixture was stirred at 140° C.

After stirring for 4 d under argon, inprocess-control via TLC shows quantitative conversion to the requested product. The reaction mixture was evaporated to a final volume of approx. 5 ml.

The crude product in toluene was filtered through a plug of silica. After evaporation of the combined product-fractions, a yellow oil was obtained (280 mg, 75%).

LC-MS measurements for the obtained compound are shown in FIG. 14.

### Preparative Example 16: N-((benzoxathiol-6-yl)propan-2-yl)-amino)propanenitrile

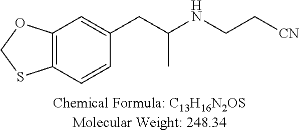 [SMILES: CC(Cc1ccc2c(c1)OCS2)NCCC#N]

Benzoxathiol-6-yl-propan-2-amine (1 mmol/197 mg) was dissolved in dichloromethane (5 ml) at 25° C. Diazabicycloundecene (1.8 mmol/0.28 ml) was added and aerated with argon. 3-Bromo-propanenitrile (1.6 mmol/0.13 ml) was added dropwise through septum. This results in a clear solution. After stirring for 24 h under argon at 25° C., inprocess-control via HPLC shows complete conversion to the requested product.

The reaction solution was diluted with dichloromethane (10 ml) and extracted with 10 ml water and 10 ml saturated saline solution. Subsequently, the organic phase was dried over some MgSO4. Subsequently, the organic phase was slowly concentrated on the rotary evaporator, yielding the raw product as a yellowish oil (250 mg).

The crude product was diluted in acetone and filtered through a plug of silica. After evaporation of the combined product-fractions, a yellowish oil was obtained (100 mg, 40%).

LC-MS measurements for the obtained compound are shown in FIG. 15.

### Preparative Example 17: N-(3,4,5-Trimethoxyphenethyl)-2-hydroxy-acetophenonimine

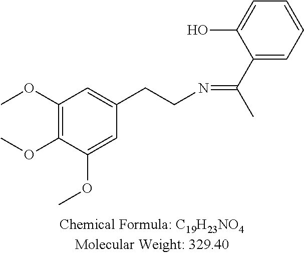 [SMILES: COc1cc(CC/N=C(\C)c2ccccc2O)cc(OC)c1OC]

3,4,5-Trimethoxyphenethylamine (1 mmol/211 mg) was diluted in toluene (10 ml) and 2-hydroxy-acetophenone (1 mmol/0.12 ml) was added. After installation of a dean-stark-apparatus, a catalytic amount of p-toluenesulfonic acid (0.03 mmol/6 mg) was added and the reaction mixture was stirred at 140° C.

After stirring for 20 h under argon, inprocess-control via TLC shows quantitative conversion to the requested product. The reaction mixture was evaporated to a final volume of approx. 2 ml.

The crude product in toluene was filtered through a plug of silica. After evaporation of the combined product-fractions, a yellow solid was obtained (460 mg, 98%).

LC-MS measurements for the obtained compound are shown in FIG. 16.

### Preparative Example 18: t-butyl-(2,5-Dimethoxy-4-methylphenethyl)carbamate

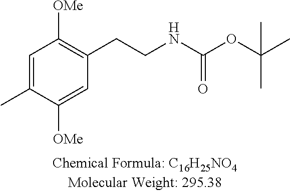 [SMILES: COc1cc(CCNC(=O)OC(C)(C)C)c(OC)cc1C]

2,5-Dimethoxy-4-methylphenethylamine (1.5 mmol/290 mg) was dissolved in tetrahydrofuran (10 ml) at 25° C. Triethylamine (2.5 mmol/0.52 ml) was added and aerated with argon. Di-tert-butyl dicarbonate (2.25 mmol/500 mg) diluted in tetrahydrofuran) was added dropwise through septum. This results in a clear colorless solution.

After stirring for 5 h under argon at 25° C., inprocess-control via HPLC shows 60% conversion to the requested product. The reaction mixture was then stirred for 2 d at 25° C.

The reaction mixture was evaporated and the remaining oil was diluted in dichloromethane. This solution was filtered through a plug of silica. After evaporation of the combined product-fractions, a colorless solid was obtained (260 mg, 58%).

LC-MS measurements for the obtained compound are shown in FIG. 17.

### Preparative Example 19: Isopropyl-(2,5-Dimethoxy-4-methylphenethyl)carbamate

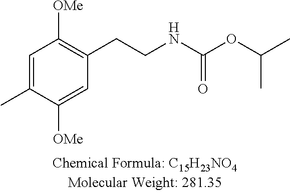 [SMILES: COc1cc(CCNC(=O)OC(C)C)c(OC)cc1C]

2,5-Dimethoxy-4-methyl-phenethylamine (1 mmol/195 mg) was dissolved in dichloromethane (8 ml) at 4° C. Triethylamine (1.2 mmol/0.17 ml) was added and aerated with argon. This results in a clear solution. Isobutyl chloroformate solution in toluene (1.2 mmol/1.2 ml) was added dropwise through septum. Upon addition, a whitish haze in the solution forms immediately. Stirring for 22 h under argon at 25° C.

Inprocess-control via TLC shows quantitative conversion to the requested product.

The grey reaction suspension was filtered through a plug of silica. After evaporation of the combined product-fractions, a colorless oil was obtained (210 mg, 75%).

LC-MS measurements for the obtained compound are shown in FIG. 18.

### Preparative Example 20: N-(2,5-Dimethoxy-4-methylphenethyl)-2-hydroxy-acetophenonimine

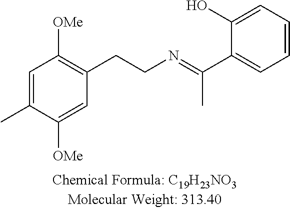 [SMILES: COc1cc(CC/N=C(\C)c2ccccc2O)c(OC)cc1C]

2,5-Dimethoxy-4-methyl-phenethylamine (2 mmol/390 mg) was diluted in toluene (10 ml) and 2-hydroxy-acetophenone (2 mmol/0.24 ml) was added. After installation of a dean-stark-apparatus, a catalytic amount of p-toluenesulfonic acid (0.06 mmol/12 mg) was added and the reaction mixture was stirred at 130° C.

After stirring for 18 h under argon, inprocess-control via TLC shows quantitative conversion to the requested product. The reaction mixture was evaporated to a final volume of approx. 10 ml.

The crude product in toluene was filtered through a plug of silica. After evaporation of the combined product-fractions, a yellow oil was obtained (290 mg, 46%).

LC-MS measurements for the obtained compound are shown in FIG. 19.

### Preparative Example 21: methyl (1-benzo[d][1,3]oxathiole-6-yl)propan-2-yl)(methyl)carbamate

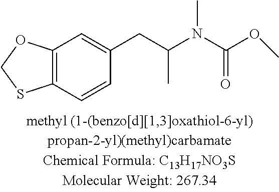 [SMILES: COC(=O)N(C)C(C)Cc1ccc2c(c1)OCS2]

Benzoxathiol-6-yl-N-methyl-propan-2-amine (0.48 mmol/100 mg) was dissolved in dichloromethane (3 ml) at 25° C. Triethylamine (0.62 mmol/0.09 ml) was added and aerated with argon. Methylchloroformate (0.53 mmol/0.04 ml) was added dropwise through septum.

After stirring for 4 h under argon at 25° C., inprocess-control via TLC shows quantitative conversion to the requested product. The reaction mixture was then diluted with dichloromethane (5 ml).

The raw product in dichloromethane was filtered through a plug of silica. After evaporation of the combined product-fractions, a colorless oil was obtained (95 mg, 74%).

LC-MS measurements for the obtained compound are shown in FIG. 23.

### Preparative Example 22: tert-butyl (1-benzo[d][1,3]oxathiole-6-yl)propan-2-yl(methyl)carbamate

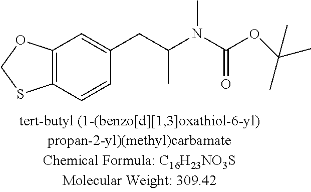 [SMILES: CC(Cc1ccc2c(c1)OCS2)N(C)C(=O)OC(C)(C)C]

Benzoxathiol-6-yl-N-methyl-propan-2-amine (0.48 mmol/100 mg) was dissolved in dichloromethane (3 ml) at 25° C. Triethylamine (0.96 mmol/0.13 ml) was added and aerated with argon. Di-tert-butyl dicarbonate (0.72 mmol/157 mg diluted in tetrahydrofuran) was added dropwise through septum.

After stirring for 4 h under argon at 25° C., inprocess-control via TLC shows quantitative conversion to the requested product. The reaction mixture was then diluted with dichloromethane (4 ml).

The raw product in dichloromethane was filtered through a plug of silica. After evaporation of the combined product-fractions, a colorless oil was obtained (120 mg, 80%).

LC-MS measurements for the obtained compound are shown in FIG. 24.

### Preparative Example 23: 2-amino-N-(1-(benzo[d][1,3]oxathiol-6-yl)propan-2-yl)-3-(1H-indol-3-yl)propanamide

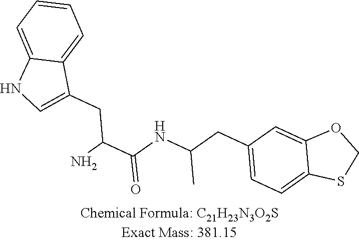 [SMILES: CC(Cc1ccc2c(c1)OCS2)NC(=O)C(N)Cc1c[nH]c2ccccc12]

HATU (2 mmol/760 mg) and Fmoc-Trp (2 mmol/850 mg) was dissolved in THF (8 ml). This yielded a white suspension. DiPEA (0.7 ml/4 mmol) was added dropwise. To this cloudy solution, Benzoxathiol-6-yl-propan-2-amine (1 mmol/195 mg) dissolved in THF (2 ml) was added. The reaction mixture was stirred for 4 h at ambient temperature. The reaction mixture was diluted with ethyl acetate (30 ml) and washed with 1N HCl, NaHCO₃-solution, water and brine. The organic phase was dried over magnesium sulfate and yielded 400 mg crude product as a light yellow oil. The crude oil was treated on a column over silica using the eluent mixture hexane/ethyl acetate in a ratio of 5:1. This yielded 190 mg of the intermediate as colorless crystals.

This Fmoc-protected intermediate was then proceeded via deprotection reaction to furnish the requested product:

The above listed intermediate (0.3 mmol/190 mg) was dissolved in THF (10 ml). To this solution, piperidine (0.6 mmol/0.06 ml) was added and the reaction mixture was stirred at ambient temperature overnight.

According to TLC/LC-MS the conversion was completed the next morning.

The reaction mixture was evaporated and the corresponding residue was chromatographed over silica using the eluent mixture hexane/ethyl acetate in a ratio of 5:1. This yielded a colorless oil (90 mg).
Retention time (HPLC): 4.7 min
UV-Maximum: 280 nm
m/z=382.00
Rf-value: 0.5 (dichlormethane/methanol 9:1)

### Preparative Example 24: 2-amino-N-(1-(benzo[d][1,3]oxathiol-6-yl)propan-2-yl)-N-methylacetamide

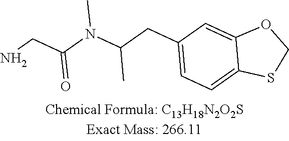 [SMILES: CC(Cc1ccc2c(c1)OCS2)N(C)C(=O)CN]

HATU (2 mmol/760 mg) and Fmoc-Gly (1.5 mmol/446 mg) was dissolved in THF (8 ml). This yielded a white suspension. DiPEA (0.7 ml/4 mmol) was added dropwise. To this cloudy solution, Benzoxathiol-6-yl-N-methyl-propan-2-amine (1 mmol/209 mg) was dissolved in THF (2 ml) was added. The reaction mixture was stirred overnight at ambient temperature.

The yellow reaction mixture was diluted with ethyl acetate (30 ml) and washed with 1N HCl, NaHCO₃-solution, water and brine. The organic phase was dried over magnesium sulfate and yielded 410 mg crude product as a light yellow oil. The crude oil was treated on a column over silica using the eluent mixture hexane/ethyl acetate in a ratio of 5:1. This yielded 150 mg of the intermediate as colorless oil.

This Fmoc-protected intermediate was then proceeded via deprotection reaction to furnish the requested product:

The above listed intermediate (0.31 mmol/150 mg) was dissolved in THF (10 ml). To this solution, piperidine (0.6 mmol/0.06 ml) was added and the reaction mixture was stirred at ambient temperature overnight.

According to TLC/LC-MS the conversion was completed the next morning.

The reaction mixture was evaporated and the corresponding residue was chromatographed over silica using the eluent mixture hexane/ethyl acetate in a ratio of 5:1.

This yielded colorless crystals (80 mg).
Retention time (HPLC): 4.1 min
UV-Maximum: 300 nm
m/z=267.00
Rf-value: 0.25 (dichlormethane/methanol 9:1)

### Preparative Example 25: 2-amino-N-(1-(benzo[d][1,3]oxathiol-6-yl)propan-2-yl)acetamide

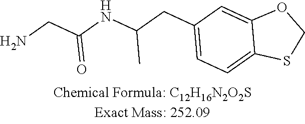 [SMILES: CC(Cc1ccc2c(c1)OCS2)NC(=O)CN]

HATU (2 mmol/760 mg) and Fmoc-Gly (1.5 mmol/446 mg) was dissolved in THF (8 ml). This yielded a white suspension. DiPEA (0.7 ml/4 mmol) was added dropwise. To this cloudy solution, Benzoxathiol-6-yl-propan-2-amine (1 mmol/195 mg) was dissolved in THF (2 ml) was added. The reaction mixture was stirred for 3.5 h at ambient temperature.

The reaction mixture was diluted with ethyl acetate (30 ml) and washed with 1N HCl, NaHCO₃-solution, water and brine. The organic phase was dried over magnesium sulfate and yielded 410 mg crude product as a yellow oil.

The crude oil was treated on a column over silica using the eluent mixture hexane/ethyl acetate in a ratio of 5:1. This yielded 210 mg of the intermediate as colorless foam.

This Fmoc-protected intermediate was then proceeded via deprotection reaction to furnish the requested product:

The above listed intermediate (0.44 mmol/210 mg) was dissolved in THF (10 ml). To this solution, piperidine (0.7 mmol/0.09 ml) was added and the reaction mixture was stirred at ambient temperature for 5.5 h.

According to TLC/LC-MS the conversion was completed.

The reaction mixture was evaporated and the corresponding residue was chromatographed over silica using the eluent mixture hexane/ethyl acetate in a ratio of 5:1.

This yielded a colorless solid (200 mg).
Retention time (HPLC): 3.9 min
UV-Maximum: 299 nm
m/z=253.00
Rf-value: 0.2 (dichlormethane/methanol 9:1)

### Preparative Example 26: Benzoxathiol-6-yl-propan-2-amine (Reference Example)

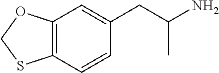

A vessel was charged with a solution of 300 mmol lithium aluminium hydride in tetrahydrofuran (300 ml). 6-(2-nitropropenyl)benzo[d][1,3]oxathiole dissolved in 100 ml tetrahydrofuran was added dropwise to this solution. During the addition, the inner temperature increased to 50° C. Thereafter the reaction mixture was stirred under reflux for 5 hours until the conversion was completed.

Then the reaction mixture was diluted with 500 ml tetrahydrofuran and cooled to 5° C. Sodium sulfate decahydrate (50 g) was added and the quenching process was left over night. The reaction mixture was filtered and the filter cake washed with tetrahydrofuran. The resulting filtrate was evaporated to yield 5.1 g crude product. The crude oil was treated on a column over 90 g silica using the eluent mixture dichloromethane/methanol in a ratio of 9:1. This yielded 3.7 g of the product as a colorless oil (47%).

LC-MS measurements for the obtained compound are shown in FIG. 28.

### Example 1: Stability Studies of Novel Phenethylamine Prodrugs in HCl and Phosphate Buffers

Three novel compounds i.e., tert-butyl (2,5-dimethoxy-4-methylphenethyl)carbamate (Preparative Example 18), tert-butyl (2,5-dimethoxyphenethyl)carbamate (Preparative Example 7), and N-(3,4,5-Trimethoxyphenethyl-2-hydroxy-acetophenon imine (Preparative Example 17) were tested for their stability in 1% hydrochloric acid (HCl) as well as in phosphate buffers at pH 7.4 and pH 8.0. These conditions were selected to provide insights into the chemical stability of compounds at a pH values similar to conditions within the human body. The diluted hydrochloric acid (pH 1) represents gastric acid within the stomach whereas the potassium phosphate buffers at pH 7.4 and pH 8.0 simulate the conditions in the human blood and small intestine, respectively.

### Methods

4 mg of each test compound was diluted in either HCl solution or phosphate buffer (0.1 M) to give a solution of 1 mg/ml. Regarding the acidic testing with hydrochloric acid, 2 ml of aqueous test compound solutions were then added to 2 ml of 0.64 M HCl, yielding a final HCl concentration of 0.32 M (pH 0.5). Since all compounds tested, represent «free bases», a minimal amount of 100-200 microliters of dimethylsulfoxide (DMSO) has been added to avoid aggregation effects during testing. Test solutions were incubated at 37° C. with continuous stirring for approximately 21 hours. Concentrations of parent compound were analyzed at various timepoints using LC-MS. Concentrations of both parent prodrug and drug liberated were expressed relative to the starting concentration of parent prodrug.

### Results

The results obtained in this experiment are shown in FIGS. 20-22.

### Conclusions

In view of the results, it has been found that all prodrugs tested displayed degradation in 1% HCl and yielded the corresponding drug. Moreover, both tert-butylcarbamates were especially susceptible to degradation by 1% HCl, showing rapid and complete chemical degradation to the corresponding phenethylamine within approximately two to four hours of testing. Degradation tests at neutral and basic conditions (pH 7.4/pH 8.0) revealed complete stability of both carbamates (2,5-dimethoxy-4-methylphenethyl)carbamate and tert-butyl (2,5-dimethoxyphenethylcarbamate) to these conditions.

Imine-prodrug (N-(3,4,5-Trimethoxyphenethyl-2-hydroxy-acetophenon imine demonstrated a more retarded degradation behavior. Due to their chemical nature, the imines show slow but significant degradation in neutral/basic conditions as well. These findings support the interpretation that the disclosed phenethylamine prodrugs are particularly suitable for tuning the drug liberation for distinct pH-values as well as specific release times.

### Example 2: Interaction Profiles of RT054, RT056 and AM176 with Human Neurotransmitter Transporters

Herein, the in vitro effects of three compounds, RT054, RT056 and prodrug AM176, on human embryonic kidney 293 (HEK293) cells, stably expressing monoamine transporters, i.e. the transporters for dopamine (DAT), norepinephrine (NET) or serotonin (SERT), in terms of their interaction profile to inhibit uptake of or elicit transporter-mediated release of transporter substrates, have been investigated. The goal of investigation was to ascertain that (i) the compounds interact with the respective targets as described above, with or without pre-treatment with hydrochloric acid, mimicking the gastric passage, and (ii) that the compounds trigger transporter-mediated efflux as known from the literature. The compound RT-056 corresponds to preparative example 11, and the compound AM176 corresponds to preparative example 22, as described herein above. The compound RT-054 corresponds to preparative example 26 (reference example)

### Methodology

### Cell Culture

Human embryonic kidney (HEK) 293 cells stably expressing human DAT, NET or SERT were maintained in humidified atmosphere (37° C., 5% CO₂) in Dulbecco's Modified Eagle Medium (DMEM), supplemented with 10% heat-inactivated fetal calf serum (FCS), streptomycin (100 μg×100 mL⁻¹) and penicillin (100 U×100 mL⁻¹). Geneticin (50 μg×mL⁻¹) was used as selection antibiotic.

### Uptake Inhibition Experiments

One day before the experiment, HEK293 cells expressing the respective transporters (DAT, NET or SERT) were seeded onto poly-D-lysine coated 96 well plates at a density of 30,000 cells per well in a final volume of 200 μL. For uptake experiments, the cell culture medium was replaced with 300 μL Krebs-HEPES buffer (KHB; 25 mM HEPES, 120 mM NaCl, 5 mM KCl, 1.2 mM CaCl₂, 1.2 mM MgSO₄, and 5 mM D-glucose, pH 7.3). Then, the buffer was replaced with various concentrations of test compounds for 5 minutes; subsequent to that, 0.1 μM [³H]5-HT (SERT), 0.1 μM [³H]dopamine (DAT), or 0.05 μM [³H]MPP⁺ (NET) in KHB was added to yield a total volume of 50 μL in the well. For determination of non-specific uptake, paroxetine was used in case of SERT (3 μM), vanoxerine (also known as GBR12909) for DAT and NET (50 μM).

Uptake was terminated after 60 s (SERT) or 180 s (DAT, NET) by washing the cells with 200 μL ice-cold KHB. Subsequently, the cells were lysed with 200 μL Ultima Gold™ XR liquid scintillation cocktail, and the amount of tritium in the cells was measured with a Wallac 1450 MicroBeta® TriLux liquid scintillation counter. Monoamine uptake data were fitted by nonlinear regression, Vₘₐₓ and Kₘ values were calculated from Michaelis Menten's least-squares fit with GraphPad Prism (Prism 9.0.2, GraphPad Software, San Diego, CA, USA).

### Release Experiments

The day prior to the assay, stably expressing HEK293 cells were seeded in a perfusion chamber of an IBIDI microfluidic chip (μ-Slide VI 0.5 Glass Bottom, ibidi GmbH). Chambers were previously coated with poly-D-lysine for 20 min at 37° C. and then cells were seeded at a density of approximately 1-10⁶ cells×mL⁻¹.

For the release experiment, cells were loaded with radioactive substrate (0.05 μM [³H]MPP⁺ (DAT, NET) or 0.08 μM [³H]5-HT (SERT) for 20 min at 37° C.) and then perfused with different solutions at constant flow rate (0.6 μL·min⁻¹). The assay included washout with Krebs-Hepes buffer (KHB, pH 7.3; 20 min) to establish a stable baseline efflux, perfusion of cells with KHB (6 min) and collection of the baseline samples, injection of control compounds (DAT, NET: S-amphetamine 10 μM, SERT: para-chloroamphetamine 3 μM) or test compounds at various concentrations for 10 min. The cells were flushed using sodium dodecyl sulfate (SDS) to retrieve the residual radioactivity (6 min). Interaction of detergent with the cells lysed the cell membranes and all the radioactive substrate was collected at the outlet.

Radioactivity was quantified with a beta-scintillation counter (PerkinElmer, Waltham, MA, USA). The released amount of tritiated substrate for each fraction was expressed as the amount of released tritium compared to the amount remaining in the cell at the end of the previous fraction.

### Test Compounds

RT054 and RT056 were tested in both uptake inhibition and release assays, and the corresponding prodrug AM176 was tested in uptake inhibition assays. AM176 was tested either as the undecomposed prodrug or after treatment with 0.5% HCl at 37° C. for 24 hours in order to simulate digestion of the prodrug by gastric acid inside the body after peroral administration.

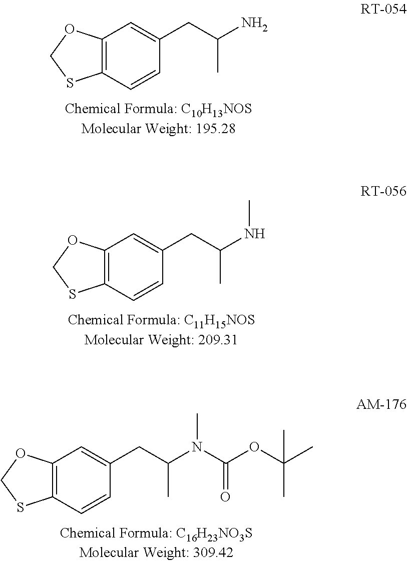 [SMILES: CC(N)Cc1ccc2c(c1)OCS2 ; CNC(C)Cc1ccc2c(c1)OCS2 ; CC(Cc1ccc2c(c1)OCS2)N(C)C(=O)OC(C)(C)C]

### Results

### RT054

In the uptake inhibition assays, RT054 shows interaction with all three monoamine transporters of interest, i.e. the compound inhibits substrate-uptake via all three transporters in a competitive manner (see FIG. 25 for details). The release assays show unanimously that the compound RT054 induces efflux in a manner comparable to MDMA (see FIG. 1 in Hilber et al., Neuropharmacology, 2005, doi: 10.1016/j.neuropharm.2005.08.008). While almost full efficacious release is observable in HEK293 cells expressing DAT and NET, the compound behaves as a partial releaser at HEK293 cells expressing SERT (see FIG. 25).

### RT056

In the uptake inhibition assays, RT056 shows interaction with all three monoamine transporters of interest, i.e. the compound inhibits substrate-uptake via all three transporters in a competitive manner (see FIG. 26). The release assays show unanimously that the compound RT056 induces efflux at all monoamine transporters of interest. While almost full efficacious release is observable in HEK293 cells expressing NET, interestingly, the compound behaves as a partial releaser at HEK293 cells expressing DAT and SERT (see FIG. 26).

### AM176

In these uptake inhibition assays, AM176 shows no interaction without pre-treatment with HCl at all, but only after pre-treatment with HCl, there is interaction with all monoamine transporters of interest, i.e. the HCl-treated compound inhibits substrate-uptake via all monoamine transporters in a competitive manner (see FIG. 27).

### CONCLUSIONS

In conclusion, due to their similarity to MDMA, the compounds RT054 and RT56 inhibit the monoamine transporters unanimously; in addition, they induce transporter-mediated efflux in their specific manner, i.e. at their specific profile. AM176 was tested in uptake inhibition assays with or without pre-treatment conditions: Without pre-treatment with HCl, the Boc-moiety prevented the compound from any transporter interaction as a result of its bulkiness. However, after treatment with HCl, which was done in order to simulate the gastric passage, the Boc-moiety is removed efficiently and the resulting compound yields uptake inhibition in a similar range to the one seen with RT054 and RT056. However, the IC₅₀-values are approximately 3 times higher than those of RT054 and RT056 (see summary in Table 1 below for details). It has been further demonstrated that under conditions mimicking physiological conditions, the prodrug AM176 retains activity similar to that of RT056 (whereby the deprotection reaction of AM176 upon HCl treatment is meant to yield RT056), thus supporting the functionality of the prodrug compounds provided in accordance with the present invention.

TABLE 1
Uptake inhibition, IC₅₀ values

| | DAT | NET | SERT |
|---|---|---|---|
| RT054 | 1.3 μM | 0.8 μM | 3.6 μM |
| RT056 | 0.9 μM | 1.8 μM | 4.5 μM |
| AM176, HCl-treated | 3.4 μM | 9.2 μM | 18.3 μM |

1. A compound of formula (I): 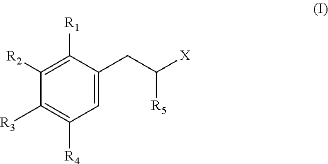
or a pharmaceutically acceptable salt thereof, wherein:
R₁ is selected from hydrogen, methyl and methoxy;
R₂ is selected from hydrogen, trifluoromethyl, methoxy and ethoxy, and
R₃ is selected from hydrogen, halogen, nitro, methyl, ethyl, n-propyl, trifluoromethyl, methoxy, ethoxy, n-propoxy, allyloxy, 2-methylallyloxy, ethylthio, n-propylthio, isopropylthio, cyclopropylmethylthio, tertbutylthio, and 2-fluoroethylthio; or
R₂ and R₃ together with the carbon atoms that carry R₂ and R₃, respectively, form a five membered ring selected from 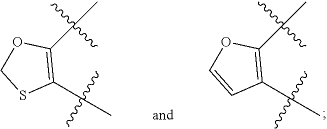 [SMILES: *C1=C(*)SCO1 ; *c1ccoc1*]
R₄ is selected from hydrogen, methyl and methoxy;
R₅ is selected from hydrogen and methyl;
X is selected from: 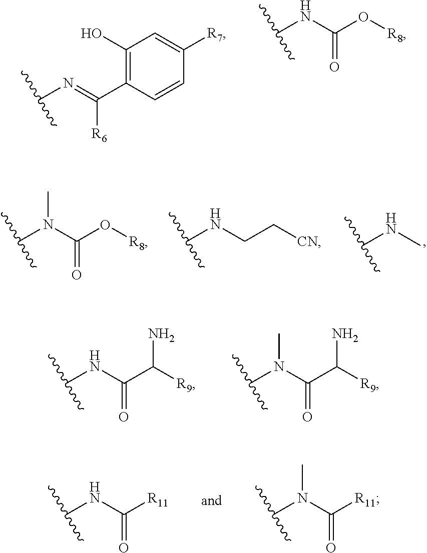
R₆ is selected from C₁₋₅ alkyl and aryl, wherein said aryl is optionally substituted with one or more groups R^{S};
R₇ is selected from methoxy and hydrogen;
R₈ is C₁₋₅ alkyl;
R₉ is selected from hydrogen, C₁₋₈ alkyl, C₂₋₈ alkenyl, C₂₋₈ alkynyl, —(C₁₋₆ alkylene)-O—R₁₀, —(C₁₋₆ alkylene)-S—R₁₀, —(C₁₋₆ alkylene)-N(R₁₀)—R₁₀, —(C₁₋₆ alkylene)-CO—R₁₀, —(C₁₋₆ alkylene)-COO—R₁₀, —(C₁₋₆ alkylene)-O—CO—(C₁₋₆ alkyl), —(C₁₋₆ alkylene)-CO—N(R₁₀)—R₁₀, —(C₁₋₆ alkylene)-N(R₁₀)—CO—(C₁₋₆ alkyl), —(C₁₋₆ alkylene)-CO—N(R₁₀)—O—R₁₀, —(C₁₋₆ alkylene)-O—CO—N(R₁₀)—R₁₀, —(C₁₋₆ alkylene)-N(R₁₀)—CO—N(R₁₀)—R₁₀, —(C₁₋₆ alkylene)-N(R₁₀)—C(═N—R₁₀)—N(R₁₀)—R₁₀, —(C₁₋₆ alkylene)-SO₃—R₁₀, —(C₀₋₆ alkylene)-carbocyclyl, and —(C₁₋₆ alkylene)-heterocyclyl, wherein the carbocyclyl group in said —(C₀₋₆ alkylene)-carbocyclyl and the heterocyclyl group in said —(C₀₋₆ alkylene)-heterocyclyl are each optionally substituted with one or more groups R^{S}, wherein said alkyl, said alkenyl, said alkynyl, and any alkylene group comprised in any of the aforementioned R₉ groups are each optionally substituted with one or more —OH, and further wherein each R₁₀ is independently selected from hydrogen and C₁₋₆ alkyl;
R₁₁ is a C₁₋₈ alkyl, C₂₋₈ alkenyl, C₂₋₈ alkynyl, carbocyclyl or heterocyclyl, wherein said alkyl, said alkenyl, said alkynyl, said carbocyclyl and said heterocyclyl are each optionally substituted with one or more R^{S}; and
each R^{S} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—CO—O—(C₁₋₅ alkyl), —(C₀₋₃ alkylene)-N(C₁₋₅ alkyl)-CO—O—(C₁₋₅ alkyl), —(C₀₋₃ alkylene)-O—CO—NH—(C₁₋₅ alkyl), —(C₀₋₃ alkylene)-O—CO—N(C₁₋₅ alkyl)-(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO₂—(C₁₋₅ alkyl), —(C₀₋₃ alkylene)-SO—(C₁₋₅ alkyl), —(C₀₋₃ alkylene)-carbocyclyl, and —(C₀₋₃ alkylene)-heterocyclyl, wherein the carbocyclyl moiety in said —(C₀₋₃ alkylene)-carbocyclyl and the heterocyclyl moiety in said —(C₀₋₃ alkylene)-heterocyclyl are each optionally substituted with one or more groups independently selected from C₁₋₄alkyl, halogen, —CN, —NO₂, —OH, —O—(C₁₋₄alkyl), —SH, —S—(C₁₋₄alkyl), —NH₂, —NH(C₁₋₄alkyl), —N(C₁₋₄alkyl)(C₁₋₄ alkyl), —COOH, —COO(C₁₋₄ alkyl), —CONH₂, —CONH(C₁₋₄ alkyl), —CON(C₁₋₄ alkyl)(C₁₋₄ alkyl), —NHCO(C₁₋₄ alkyl) and —N(C₁₋₄alkyl)-CO(C₁₋₄alkyl);
wherein if X is 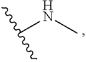 [SMILES: *NC]
then R₁ is hydrogen, R₂ and R₃ together with the carbon atoms that carry R₂ and R₃, respectively, form a five membered ring being 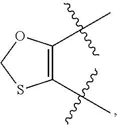 [SMILES: *C1=C(*)SCO1]
R₄ is hydrogen, and R₅ is methyl;
wherein if X is 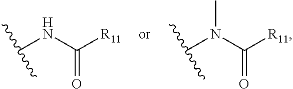
and if R₁₁ is a C₁₋₈ alkyl, C₂₋₈ alkenyl, C₂₋₈ alkynyl, or carbocyclyl, wherein said alkyl, said alkenyl, said alkynyl and said carbocyclyl are each optionally substituted with one or more R^{S}, then R₁ is hydrogen, R₂ is methoxy, R₃ is methoxy, R₄ is methoxy, and R₅ is hydrogen.

2. The compound of claim 1, wherein R₁ is selected from hydrogen and methoxy.

3. The compound of claim 1 or 2, wherein R₂ is selected from hydrogen, trifluoromethyl, methoxy and ethoxy, and R₃ is selected from hydrogen, halogen, nitro, methyl, ethyl, propyl, trifluoromethyl, methoxy, ethoxy, n-propoxy, allyloxy, 2-methylallyloxy, ethylthio, n-propylthio, isopropylthio, cyclopropylmethylthio, tertbutylthio, and 2-fluoroethylthio.

4. The compound of any one of claims 1 to 3, wherein R₂ is selected from hydrogen and methoxy, and R₃ is selected from hydrogen, halogen, methyl, ethyl, propyl, trifluoromethyl, methoxy, and ethoxy.

5. The compound of claim 1 or 2, wherein R₂ and R₃ together with the carbon atoms that carry R₂ and R₃, respectively, form a group 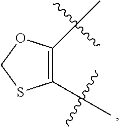 [SMILES: *C1=C(*)SCO1]
wherein the carbon atom carrying R₃ is connected to the S atom in said group

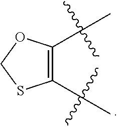 [SMILES: *C1=C(*)SCO1]

6. The compound of any one of claims 1 to 5, wherein R₄ is selected from hydrogen and methoxy.

7. The compound of any one of claims 1 to 6, wherein X is selected from:

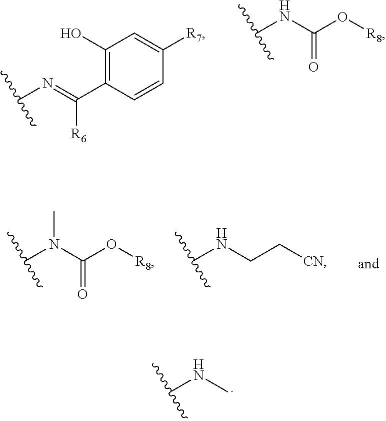

8. The compound of any one of claims 1 to 7, wherein X is selected from:

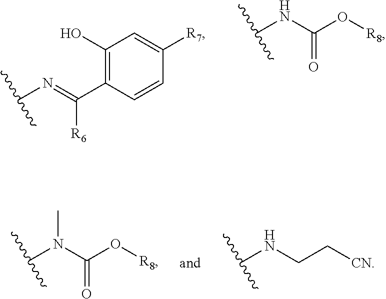

9. The compound of any one of claims 1 to 8, wherein X is selected from:

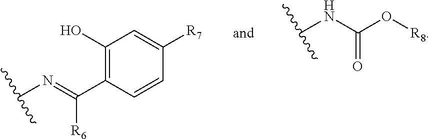

10. The compound of any one of claims 1 to 9, wherein the compound is a compound of formula (II): 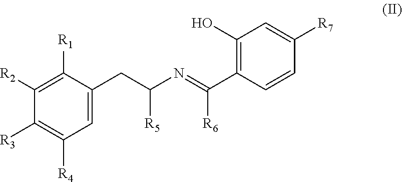
or a pharmaceutically acceptable salt thereof;
preferably wherein R₆ is selected from methyl, phenyl and 4-chloro-phenyl.

11. The compound of any one of claims 1 to 9, wherein the compound is a compound of formula (III): 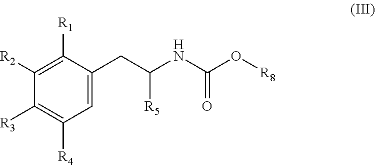
or a pharmaceutically acceptable salt thereof;
preferably wherein R₈ is selected from ethyl, isopropyl, isobutyl, tert-butyl and neopentyl.

12. The compound of any one of claims 1 to 7, wherein the compound is a compound of formula (IV): 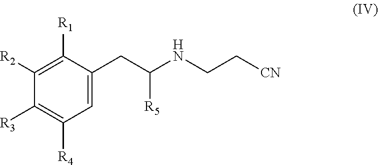
or a pharmaceutically acceptable salt thereof.

13. The compound of any one of claims 1 to 6, wherein the compound is a compound of formula (VII) 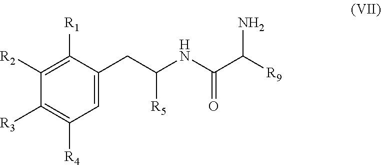
or a pharmaceutically acceptable salt thereof;
preferably wherein R₉ is hydrogen or —CH₂-(1H-indol-3-yl).

14. The compound of any one of claims 1 to 6, wherein the compound is a compound of formula (VIII): 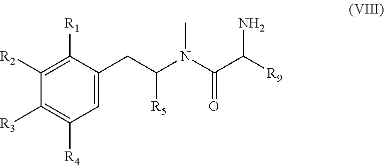
or a pharmaceutically acceptable salt thereof;
preferably wherein R₉ is hydrogen or —CH₂-(1H-indol-3-yl).

15. The compound of any one of claims 1 to 6, wherein R₁₁ is a heterocyclyl, wherein said heterocyclyl is attached via a ring carbon atom that is directly adjacent to a ring nitrogen atom, and further wherein said heterocyclyl is optionally substituted with one or more groups R^{S}.

16. The compound of claim 1, wherein said compound is selected from: 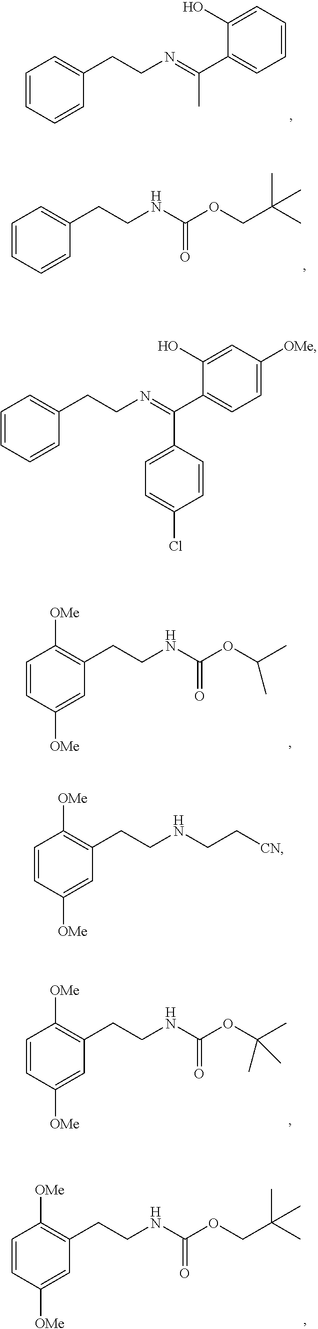 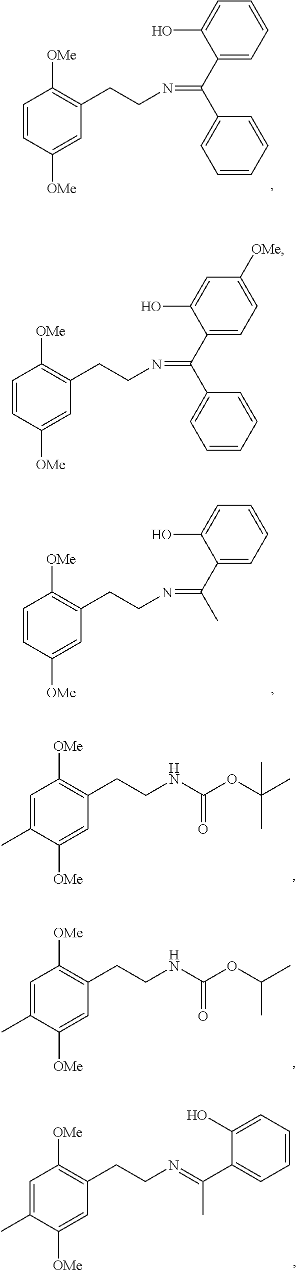 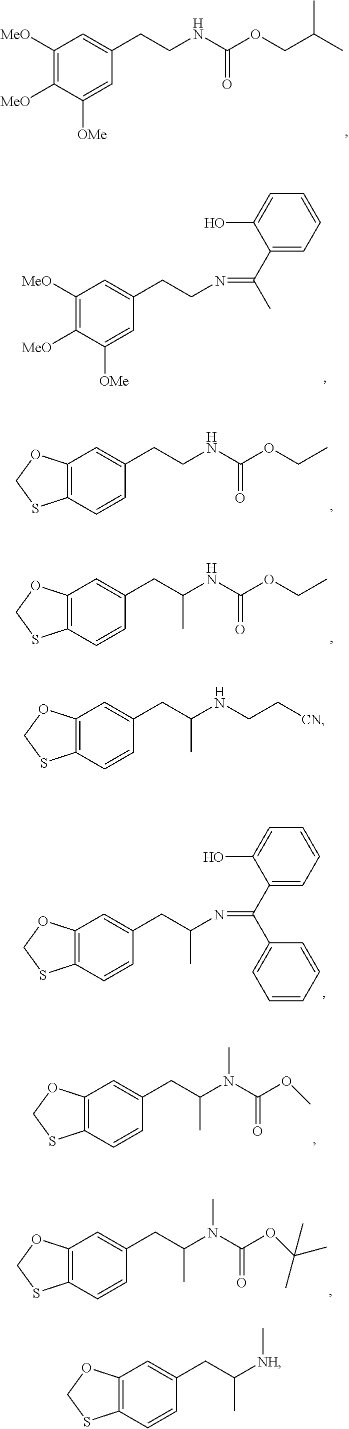 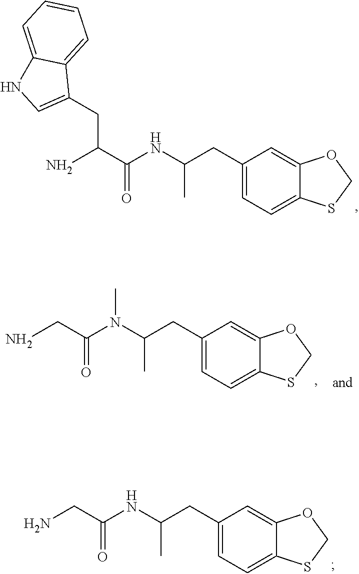
or a pharmaceutically acceptable salt of any one of the above-depicted compounds.

17. A pharmaceutical composition comprising a compound of any one of claims 1 to 16 and optionally a pharmaceutically acceptable excipient.

18. The compound of any one of claims 1 to 16 or the pharmaceutical composition of claim 17 for use as a medicament.

19. The compound of any one of claims 1 to 16 or the pharmaceutical composition of claim 17 for use in the treatment of a serotonin 5-HT_{2A} receptor associated disease/disorder.

20. The compound for use of claim 19 or the pharmaceutical composition for use of claim 19, wherein said serotonin 5-HT_{2A} receptor associated disease/disorder is an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, Alzheimer's disease or dementia.
